(19) United States
(12) Patent Application Publication
(10) Pub. No.: US 2016/0333411 A1
(43) Pub. Date: Nov. 17, 2016

(54) METHODS OF WEIGHT ANALYSIS AND USES THEREOF

(71) Applicant: Ruthie Harper, Austin, TX (US)

(72) Inventor: Ruthie Harper, Austin, TX (US)

(21) Appl. No.: 15/112,125

(22) PCT Filed: Jan. 16, 2015
(86) PCT No.: PCT/US15/11819

§ 371 Date: Jul. 15, 2016

Related U.S. Application Data
(60) Provisional application No. 61/928,851, filed on Jan. 17, 2014.

(51) Int. Cl. C12Q 1/68; G06F 19/28; G06F 19/00; G06F 19/22

(52) U.S. Cl. CPC C12Q 1/6883; G06F 19/22; G06F 19/28; G06F 19/3431; C12Q 2600/156; C12Q 2600/118

(57) ABSTRACT

Disclosed herein are weight analysis methods and assays, which objectively identify biological strengths and weaknesses in a patient's genetic coding that affect their weight. The generated results can be utilized to develop a personalized weight and nutritional regimen to regulate weight and prevent, reduce and treat weight disorders and diseases. In some embodiments, a method of characterizing a subject's weight is provided which includes generating a personalized weight profile by determining a subject's genetic potential in at least one area of weight health by analyzing one or more weight health-associated single nucleotide polymorphisms (SNPs) or other genetic markers associated with the particular area of weight health being assessed in a sample obtained from the subject. The generated weight profile reveals the subject's genetic strengths, weaknesses and/or risks related to the one or more areas of weight health thereby allowing a personalized weight and/or nutritional regimen to be developed and implemented.

### CROSS REFERENCE TO RELATED APPLICATION

This application claims the benefit under 35 U.S.C. 119(e) of the earlier filing date of U.S. Patent Application No. 61/928,851 filed on Jan. 17, 2014, which is incorporated herein by reference in its entirety.

### FIELD

This disclosure relates to the field of weight management, more specifically to methods and assays for using single nucleotide polymorphisms (SNPs) or other genetic markers associated with weight regulation, such as weight loss or gain, to allow personalized weight control and maintenance of a healthy BMI, including but not limited to nutritional/diet/food products, supplements, food alternatives (shakes, bars, snacks), behavioral programs, nutritional or dietary eating program or regimen recommendations and/or exercise regimens to be developed and implemented.

### BACKGROUND

Many methods are currently used to control weight, such as various dietary and exercise regimens. Currently, methods and products used to regulate weight gain or loss are generally designed around caloric addition or restriction from food and decreased or increased caloric expenditure through exercise and are administered based upon the physically displayed symptoms of the individual. Current methods for weight control do not focus on determining the underlying causes of such conditions or changes in weight. The human genome revealed unique genetic predispositions that differ from one individual to the next and revealed how these differences may influence biological function that contribute to ideal body weight. Without considering underlying or causative factors and genetic predispositions involved in weight gain or loss, the treatment often is not as effective as it could be, for both short and long-term weight control. Specifically, the undesired alteration in weight has already developed when treatment is implemented if treatment is not commenced or modified until physical symptoms are displayed and the approach to deal with same is generalized and typically involves caloric modification only. Programs designed to optimize weight are less than optimally successful because they are not designed or personalized for each individual based on the individuals genetic strengths and weaknesses and the impact of these factors on ideal weight. A personalized approached based on each individuals genetic predispositions is not currently utilized to achieve short or long term weight success. A need remains for methods to determine a subject's overall weight profile and propensity toward deleterious weight changes by identifying factors that are unique to the individual, including genetic factors, which contribute to weight gain and loss should be used so that steps can be taken to prevent, reduce or inhibit weight conditions, disorders or diseases.

### SUMMARY

Disclosed herein are weight analysis methods and assays, which objectively identify biological strengths and weaknesses in a patient's genetic coding that can affect their body weight. The generated results can then be utilized to develop a personalized weight control, nutritional/diet/food/eating programs, supplements, food alternatives (shakes, bars, snacks), behavior modification and/or exercise regimen or protocol which can then be implemented to prevent, reduce and treat undesired fluctuations in weight or weight related disorders and/or diseases. The disclosed methods utilize single nucleotide polymorphisms (SNPs) or other genetic markers associated with weight loss or gain to develop a personalized weight control, nutritional, behavior and/or exercise regimen. In some embodiments, a method of characterizing a subject's weight is provided which includes generating a personalized weight profile. In some examples, generating a personalized weight profile includes determining a subject's genetic potential in at least one area of weight health by analyzing one or more weight health-associated single nucleotide polymorphisms (SNPs) associated with the particular area of weight health being assessed in a sample obtained from the subject. In some examples, a subject's genetic potential is determined by analyzing one or more weight health-associated SNPs in a sample obtained from the subject, in some examples, the one or more weight health-associated SNPs includes one or more of obesity or weight gain or loss related, carbohydrate, fat, protein and/or alcohol or caffeine utilization or metabolism, leptin, ghrelin and hunger, satiety or craving SNP's, blood sugar regulation and stabilization, dopamine or neurochemistry, caloric utilization and exercise SNPs or any combination thereof. The generated weight profile reveals the subject's genetic strengths, weaknesses and/or risks related to the one or more areas of weight health thereby allowing a personalized weight, nutritional/diet/food/eating program, supplements, food alternatives (shakes, bars, snacks), behavioral and or exercise regimen to be developed and implemented.

The foregoing and other features and advantages of the disclosure will become more apparent from the following detailed description.

### DETAILED DESCRIPTION OF SEVERAL EMBODIMENTS

### I. Introduction

The Human Genome Project was one of the greatest accomplishments in scientific history as it laid out the entire base code for the human genome. Following its completion in 2003, scientists began finding human population variations in the base code and began probing to understand what these variations meant. Issues raised have included what encompasses these variations, whether they are mutations, and what, if anything is there biological significance.

Certain variations were found with consistent frequencies, indicating that they were not simple mutations but rather had biological significance in human function. This was supported when scientists determined that these variations had been encoded into the genetic messaging and had been passed down through generations obtaining certain frequencies in the DNA coding.

These variations were found to be single nucleotide polymorphisms (SNPs) or differences in the base code pattern in one person's DNA compared to that of another. Scientists began an exhaustive research project to create a data bank of SNP's, determining where SNP's occur in the DNA code, with what frequency, and what they coded for, if anything, in contributing to differences in human body functions. “Active” SNPs were found to be located in coding portions of the DNA that changed biological function, giving credence to the conclusion that active SNPs serve a purpose in the body, unlike random and functionally irrelevant mutations.

Further research found that certain SNPs occurred and survived in human DNA coding for Darwinian-type reasons—they supported both function and survival in certain populations. This answered the question as to why they became encoded in DNA and passed down from one generation to the next. Herein is disclosed personalized weight, nutritional, exercise or a combination thereof regimens and protocols based upon identifying SNPs or other genetic markers pertinent to the regulation of weight and utilizing such SNPs or other genetic markers to determine a subject's weight genetic potential. The generated weight profile reveals the subject's genetic strengths, weaknesses and/or risks related to weight alterations thereby allowing one or more of the disclosed weight, nutritional, /diet/food, supplements, food alternatives (shakes, bars, snacks), behavioral and/or exercise protocols/treatments to be developed and provided.

Although the methods and assays described below primarily concern used of SNPs as the genetic marker it is contemplated that any genetic marker such as RFLP (or Restriction fragment length polymorphism), SSLP (or Simple sequence length polymorphism), AFLP (or Amplified fragment length polymorphism), RAPD (or Random amplification of polymorphic DNA), VNTR (or Variable number tandem repeat), SSR Microsatellite polymorphism, (or Simple sequence repeat), STR (or Short tandem repeat), SFP (or Single feature polymorphism), DArT (or Diversity Arrays Technology), and RAD markers (or Restriction site associated DNA markers) or any other genetic marker may be used in such methods and assays.

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). As used herein and in the appended claims, the singular forms “a”, “an”, and “the” include plural referents unless the context clearly dictates otherwise. Unless defined otherwise, all technical and scientific terms used herein have the same meaning as commonly understood to one of ordinary skill in the art to which this disclosure belongs. “Comprising” means “including.” All publications, patent applications, patents, and other references mentioned herein are incorporated by reference in their entirety.

Blood sugar: The blood sugar concentration or blood glucose level is the amount of glucose (sugar) present in the blood of a human or animal. The body naturally tightly regulates blood glucose levels as a part of metabolic homeostasis.

The mean normal blood glucose level in humans is about 5.5 mM (5.5 mmol/L or 100 mg/dL, i.e. milligrams/deciliter); however, this level fluctuates throughout the day. Glucose levels are usually lowest in the morning, before the first meal of the day (termed “the fasting level”), and rise after meals for an hour or two by a few millimolar. The normal blood glucose level (tested while fasting) for non-diabetics, should be between 70 and 100 milligrams per deciliter (mg/dL). Blood sugar levels for those without diabetes and who are not fasting should be below 125 mg/dL. The blood glucose target range for diabetics, according to the American Diabetes Association, should be 90-130 (mg/dL) before meals and less than 180 mg/dL after meals (as measured by a blood glucose monitor).

Blood sugar levels outside the normal range may be an indicator of a medical condition. A persistently high level is referred to as hyperglycemia; low levels are referred to as hypoglycemia. Diabetes mellitus is characterized by persistent hyperglycemia from any of several causes, and is the most prominent disease related to failure of blood sugar regulation.

Body Mass Index (BMI): A number calculated from a person's weight and height. BMI is an indicator of body fatness for most people and is used to screen for weight categories that may lead to health problems. BMI is calculated the same way for both adults and children. For adults 20 years old and older, BMI is interpreted using standard weight status categories that are the same for all ages and for both men and women. For children and teenagers the interpretation of BMI is both age- and sex-specific.

BMI calculation is based on the following formulas:

| Measurement | |
|---|---|
| Units | Formula and Calculation |
| Kilograms | Formula: weight (kg)/[height (m)]² |
| and meters | With the metric system, the formula for BMI is weight |
| (or | in kilograms divided by height in meters squared. Since |
| centimeters) | height is commonly measured in centimeters, divide |
| | height in centimeters by 100 to obtain height in meters. |
| | Example: Weight = 68 kg, Height = 165 cm (1.65 m) |
| | Calculation: 68 ÷ (1.65)² = 24.98 |
| Pounds | Formula: weight (lb.)/[height (in)]² × 703 |
| and inches | Calculate BMI by dividing weight in pounds (lbs.) by |
| | height in inches (in) squared and multiplying by a |
| | conversion factor of 703. |
| | Example: Weight = 150 lbs., Height = 5′5″ (65″) |
| | Calculation: [150 ÷ (65)²] × 703 = 24.96 |

The standard weight status categories associated with BMI ranges for adults are shown in the following table.

| BMI | Weight Status |
|---|---|
| Below 18.5 | Underweight |
| 18.5-24.9 | Normal |
| 25.0-29.9 | Overweight |
| 30.0 and Above | Obese |

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal (termination codon). The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a protein, or a fragment thereof, encompasses both the sense strand and its reverse complement. Thus, for instance, it is appropriate to generate probes or primers from the reverse complement sequence of the disclosed nucleic acid molecules.

Dopamine (or 3,4-dihydroxyphenethylamine): A neurotransmitter in the catecholamine and phenethylamine families. Its name derives from its chemical structure: it is an amine that is formed by removing a carboxyl group from a molecule of L-DOPA. Dopamine has a variety of roles in the brain and throughout the body, including regulation of appetite and growth hormone. The dopamine D2 receptor gene plays a role in regulation of weight (obesity)—D2 receptor antagonists enhance appetite and D2 receptor agonists suppress appetite.

Gene: A segment of DNA that contains the coding sequence for a protein, wherein the segment may include promoters, exons, introns, and other untranslated regions that control expression.

Genetic marker: A gene or DNA sequence with a known location on a chromosome that can be used to identify individuals or species. It can be described as a variation (which may arise due to mutation or alteration in the genomic loci) that can be observed. A genetic marker may be a short DNA sequence, such as a sequence surrounding a single base-pair change (single nucleotide polymorphism, SNP), or a long one, like minisatellites. Some commonly used types of genetic markers are RFLP (or Restriction fragment length polymorphism), SSLP (or Simple sequence length polymorphism), AFLP (or Amplified fragment length polymorphism), RAPD (or Random amplification of polymorphic DNA), VNTR (or Variable number tandem repeat), SSR Microsatellite polymorphism, (or Simple sequence repeat), SNP (or Single nucleotide polymorphism), STR (or Short tandem repeat), SFP (or Single feature polymorphism), DArT (or Diversity Arrays Technology), and RAD markers (or Restriction site associated DNA markers). Molecular genetic markers can be divided into two classes a) biochemical markers which detect variation at the gene product level such as changes in proteins and amino acids and b) molecular markers which detect variation at the DNA level such as nucleotide changes: deletion, duplication, inversion and/or insertion. Disclosed herein are methods and assays for using genetic markers, such as, but not limited to, single nucleotide polymorphisms (SNPs) associated with weight regulation, such as weight loss or gain, to allow personalized weight control and maintenance of a healthy BMI.

Genetic predisposition: Susceptibility of a subject to a particular condition or disease, such as a weight condition or related disease. Detecting a genetic predisposition can include, but does not necessarily include, detecting the presence of the condition or disease itself, such as but not limited to an early stage of the condition or disease process. Detecting a genetic predisposition also includes detecting the risk of developing the condition or disease, and determining the susceptibility of that subject to developing the condition or disease or to having a poor prognosis for the disease. Thus, if a subject has a genetic predisposition to a weight disorder or disease they do not necessarily develop the disorder or disease but are at risk for developing the disorder or disease.

Genomic target sequence: A sequence of nucleotides located in a particular region in the human genome that corresponds to one or more specific genetic abnormalities, such as a nucleotide polymorphism, a deletion, an insertion, or an amplification. The target can be for instance a coding sequence; it can also be the non-coding strand that corresponds to a coding sequence. The target can also be a non-coding sequence, such as an intronic sequence.

Ghrelin: A 28 amino acid hunger-stimulating peptide and hormone that is produced mainly by P/D1 cells lining the fundus of the human stomach and epsilon cells of the pancreas. Ghrelin together with obestatin is produced from cleavage of the ghrelin/obestatin prepropeptide (also known as the appetite-regulating hormone or growth hormone secretagogue or motilin-related peptide) which in turn is encoded by the GHRL gene. Ghrelin receptors are expressed in a wide variety of tissues, including the pituitary, stomach, intestine, pancreas, thymus, gonads, thyroid, and heart. The diversity of ghrelin receptor locations suggests ghrelin has diverse biological functions.

Ghrelin has been linked to inducing appetite and feeding behaviors. Ghrelin levels increase before meals and decrease after meals. It is considered the counterpart of the hormone leptin, produced by adipose tissue, which induces satiation when present at higher levels. Studies have shown that ghrelin levels are negatively correlated with weight.

Isolated: An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Leptin: A 16-kDa adipokine that plays a key role in regulating energy intake and expenditure, including appetite and hunger, metabolism, and behavior. It is an adipose-derived hormones and functions by binding to the leptin receptor. Leptin acts on the brain to regulate food intake and body weight. In particular, leptin inhibits appetite.

Locus: A location on a chromosome or DNA molecule corresponding to a gene or a physical or phenotypic feature, where physical features include polymorphic sites.

Polymorphism: A variation in a gene sequence. The polymorphisms can be those variations (DNA sequence differences) which are generally found between individuals or different ethnic groups and geographic locations which, while having a different sequence, produce functionally equivalent gene products. Typically, the term can also refer to variants in the sequence which can lead to gene products that are not functionally equivalent. Polymorphisms also encompass variations which can be classified as alleles and/or mutations which can produce gene products which may have an altered function. Polymorphisms also encompass variations which can be classified as alleles and/or mutations which either produce no gene product or an inactive gene product or an active gene product produced at an abnormal rate or in an inappropriate tissue or in response to an inappropriate stimulus. Alleles are the alternate forms that occur at the polymorphism.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation.

In the instant application “polymorphism” refers a traditional definition, in that the definition “polymorphism” means that the minor allele frequency must be greater than at least 1%.

A “single nucleotide polymorphism (SNP)” is a single base (nucleotide) polymorphism in a DNA sequence among individuals in a population. Typically in the literature, a single nucleotide polymorphism (SNP) may fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed “synonymous” (sometimes called a silent mutation)—if a different polypeptide sequence is produced they are “nonsynonymous”. A nonsynonymous change may either be missense or “nonsense”, where a missense change results in a different amino acid, while a nonsense change results in a premature stop codon.

Obesity: A medical condition in which excess body fat has accumulated to the extent that it may have an adverse effect on health, leading to reduced life expectancy and/or increased health problems. Humans are considered obese when their body mass index (BMI), a measurement obtained by dividing a person's weight in kilograms by the square of the person's height in meters, exceeds 30 kg/m². In children, a healthy weight varies with age and sex. Obesity in children and adolescents is defined not as an absolute number but in relation to a historical normal group, such that obesity is a BMI greater than the 95th percentile. Obesity increases the likelihood of various diseases, particularly cardiovascular disease, diabetes mellitus type 2, obstructive sleep apnea, certain types of cancer, asthma and osteoarthritis. Obesity is most commonly caused by a combination of excessive food energy intake, lack of physical activity, and genetic susceptibility, endocrine disorders, medications or psychiatric illness.

Dieting and physical exercise are the mainstays of treatment for obesity. Diet quality can be improved by reducing the consumption of energy-dense foods such as those high in fat and sugars, and by increasing the intake of dietary fiber. Anti-obesity drugs may be taken to reduce appetite or inhibit fat absorption together with a suitable diet. If diet, exercise and medication are not effective, a gastric balloon may assist with weight loss, or surgery may be performed to reduce stomach volume and/or bowel length, leading to earlier satiation and reduced ability to absorb nutrients from food.

Probes and primers: A probe comprises an isolated nucleic acid capable of hybridizing to a target nucleic acid. A detectable label or reporter molecule can be attached to a probe or primer. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example in Sambrook et al. (In Molecular Cloning: A Laboratory Manual, CSHL, New York, 1989) and Ausubel et al. (In Current Protocols in Molecular Biology, John Wiley & Sons, New York, 1998).

In a particular example, a probe includes at least one fluorophore, such as an acceptor fluorophore or donor fluorophore. For example, a fluorophore can be attached at the 5′- or 3′-end of the probe. In specific examples, the fluorophore is attached to the base at the 5′-end of the probe, the base at its 3′-end, the phosphate group at its 5′-end or a modified base, such as a T internal to the probe.

Probes are generally at least 15 nucleotides in length, such as at least 15, at least 16, at least 17, at least 18, at least 19, least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50 at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, or more contiguous nucleotides complementary to the target nucleic acid molecule, such as 20-70 nucleotides, 20-60 nucleotides, 20-50 nucleotides, 20-40 nucleotides, or 20-30 nucleotides.

Primers are short nucleic acid molecules, for instance DNA oligonucleotides are 10 nucleotides or more in length, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule.

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 15, 20, 25, 30, 35, 40, 45, 50, 55, 60, 65, 70 or more consecutive nucleotides. In particular examples, a primer is at least 15 nucleotides in length, such as at least 15 contiguous nucleotides complementary to a target nucleic acid molecule. Particular lengths of primers that can be used to practice the methods of the present disclosure include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, or more contiguous nucleotides complementary to the target nucleic acid molecule to be amplified, such as a primer of 15-70 nucleotides, 15-60 nucleotides, 15-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

Nucleic acid probes and primers can be readily prepared based on the nucleic acid molecules provided herein. It is also appropriate to generate probes and primers based on fragments or portions of these disclosed nucleic acid molecules, for instance regions that encompass the identified polymorphisms of interest. PCR primer pairs can be derived from a known sequence by using computer programs intended for that purpose such as Primer (Version 0.5, © 1991, Whitehead Institute for Biomedical Research, Cambridge, Mass.) or PRIMER EXPRESS® Software (Applied Biosystems, AB, Foster City, Calif.).

Sample: A sample, such as a biological sample, is a sample obtained from a subject. As used herein, biological samples include all clinical samples useful for establishing a weight profile for a subjects, including, but not limited to, cells, tissues, and bodily fluids (such as saliva); biopsied or surgically removed tissue, including tissues that are, for example, unfixed, frozen, fixed in formalin and/or embedded in paraffin; tears; skin scrapes; or surface washings. In a particular example, a sample includes cells collected by using a swab or by an oral rinse.

Subject: Living multi-cellular vertebrate organisms, a category that includes human and non-human mammals (such as laboratory or veterinary subjects).

Underweight: A term used herein to describe a human whose body weight is considered too low to be healthy. The definition usually refers to people with a BMI of under 18.5 or a weight 15% to 20% below that normal for their age and height group.

### III. Weight Analysis Methods

Disclosed herein are weight analysis methods and assays which objectively identify biological strengths and weaknesses in a patient's genetic coding that can affect their body weight. The disclosed methods and assays utilize a subject's unique genetic information to identify the genetic potential of the subject in at least one area of weight health and thus reveal the subject's genetic predisposition or potential for certain weight conditions and/or disorders. In each area of weight health, a score is generated (which may range from 0.00-1.00, with 1.00 being the optimal result or have some other numerical scoring) by analyzing a sample of the subject's DNA for genetic variations (or SNPs) that affect normal gene functioning. The test subjects score for each area of weight health is determined by an algorithm that weighs optimally functioning versus non-optimally functioning SNPs or other genetic marker and uses that algorithm to calculate the cumulative effect on the combined SNP's or genetic markers on that area of weight health. The scores are determined by giving each SNP or genetic marker a numerical value based on such things as but not limited to population frequency. These scores for each test are provided to compare the individual's score against an ideal score of 1 in order to determine risk or probability for each category of weight health. By doing this these scores and the detailed results present a personalized and highly preventive approach to controlling body weight, giving a subject the opportunity for the earliest and best intervention possible before visible signs of alterations in weight appear, and better short term and long term results at generating a program that will provide them with ideal weight.

In some examples, a personalized weight profile is generated by the following process:
1. Obtain genomic DNA sample, such as from saliva, cheek swab, skin, or other tissue;
2. Isolate the genomic DNA from sample;
3. Verify the DNA quality and concentration;
4. Amplify the DNA;
5. Use the DNA as a template to detect/amplify the SNP regions for sequence determination;
6. Sequence the SNP regions to determine alleles/variants present;
7. Input the raw data into a processing database to verify sequences in human population;
8. Perform quality control check and input subject data into algorithms and group data analysis to determine scores by weight health category; and
9. Perform additional quality control check and generate final, user-friendly report for individual.
   It is contemplated that one or more of these steps may be repeated and/or omitted. It is also contemplated that the order of the steps may be altered.

In some examples, a DNA report is statistically tabulated and electronically created through a computer system. For example, this DNA report may include one or more of the following:

A list of the overall risk ratings such as high, medium, or low under one or more categories of weight health, such as one, two, three, four, five, six, seven eight or more, including up to 8 or greater than 8 categories of weight health;

A description of the biological effects on weight health, such as one, two, three, four, five, six, seven, eight or more, such as up to 8 or greater than 8 categories of weight health;

A page for each of the up to 8 or greater than 8 categories describing the category in greater detail and listing the marker-specific results for the individual is created. A rating system may include the following:
a) A value of 0.1 may be assigned to a result of homozygous for the non-ideal allele at the at risk SNP location (non-ideal function), which indicates high risk and is represented by a deficient rating;
b) A value of 0.50 may be assigned to a result of heterozygous for the non-ideal allele at the at risk SNP location, which indicates medium risk and is represented by a sub-normal rating;
c) A value of 1.00 may be assigned to a result of homozygous for the ideal allele at the at risk SNP location, which indicates low risk and is represented by a normal rating;
d) A computer program/algorithm calculates a numerical value for each category of weight health that may be based on the average of the numbers provided for each SNP contained in a weight health category and generates an overall numerical rating;
e) High risk may be considered an average numerical value of 0.1-0.5. Medium risk may be considered to be a numerical average of 0.5-0.75 and low risk may be considered to be a numerical value of 0.75-1.0; and
f) Marker specific results may be listed by SNP and may include:
   The affected gene or marker
   The chromosomal location
   The ideal genotype
   The actual genotype
   The rating.
      In some examples, specific nutritional/diet/food products, supplements, food alternatives (shakes, bars, snacks), behavioral or lifestyle programs, nutritional or dietary eating program or regimen recommendations and/or exercise regimen recommendations are created according to a program based on each individual's report. Foundational products may be recommended for everyone and these do not represent the unique product recommendations made for the individual based on their genetic results. These products are relevant; however, in that they set the stage for optimal results for personalized weight health recommendations, based on each individual's unique DNA report. These foundational products may include:

a. multivitamin

b. antioxidants

c. essential fatty acids

In some examples, one or more program and/or product personalized recommendations are made based on the individual's high and medium risk or priority category results and may include: nutritional/diet/food/eating programs, supplements, food alternatives (shakes, bars, snacks), behavior or lifestyle modification and/or exercise regimen or protocols. These recommendations address the individual strengths and weaknesses as determined by their DNA report.

The determination as to which products will be custom recommended may be based on an algorithmic protocol that matches the ingredients in the products to the weakness that the person's DNA analysis reveals as identified in their medium or high risk or priority categories. This algorithm may be programmed into a computer system that generates the DNA report. High risk or priority categories provide a guideline by which the individual may achieve the greatest opportunity to improve their weight health. For instance, if a person is determined to be medium or high risk or priority for hunger and satiety, nutritional/diet/food products, supplements, food alternatives (shakes, bars, snacks), behavioral or lifestyle programs, nutritional Or dietary eating program or regimen recommendations and/or exercise regimens will be recommended that support healthy hunger and satiety in order to support healthy weight. The nutritional/diet/food/eating programs, supplements, food alternatives (shakes, bars, snacks), behavior and lifestyle modification and/or exercise regimen or protocols that support healthy hunger and satiety will be selected and put into nutritional/diet/food/eating programs, supplements, mod alternatives (shakes, bars, snacks), behavior modification lifestyle and/or exercise regimens or protocols based on clinical research that supports the clinical effectiveness of the nutritional/diet/food/eating programs, supplements, food alternatives (shakes, bars, snacks), behavior and lifestyle modification and/or exercise regimen or protocols for the category to which it applies. Nutritional/diet/food/eating programs, supplements, food alternatives (shakes, bars, snacks), behavior lifestyle modification and/or exercise regimens or protocols may be supported by clinical research regarding the effectiveness of the nutritional/diet/food/eating programs, supplements, food alternatives (shakes, bars, snacks), behavior, lifestyle modification and/or exercise regimen or protocol as identified in both peer-reviewed literature as well as clinical studies. These human clinical studies may be validated and supported for effectiveness based on well-controlled human trials. In some examples, product recommendations will be organized into high and medium risk or priority categories on the report for ease of understanding and product or program selection by the individual. In some examples, if a product is already listed under a high-risk or priority category, it will not be relisted under a medium risk category as it is already selected by the algorithm and recommended. In some examples, nutritional/diet/food/eating programs, supplements, food alternatives (shakes, bars, snacks), behavior, lifestyle modification and/or exercise regimen or protocol will be upgraded and changed on a regular basis based on the current science and literature as new nutritional/diet/food/eating programs, supplements, food alternatives (shakes, bars, snacks), behavior, lifestyle modification and/or exercise regimens or protocols with better effectiveness become available in the marketplace.

In some examples, following the formulation process and algorithmic recommendations above, nutritional/diet/food/eating programs, supplements, food alternatives (shakes, bars, snacks), behavior, lifestyle modification and/or exercise regimen or protocol may be clinically used and evaluated in a clinical practice to ensure that the effectiveness, and client satisfaction are of the highest standards possible.

It is contemplated that disclosed products and programs may be distributed into the marketplace through various distribution models which may include direct to consumer, business to business, direct sales through a multi-level marketing program, television/infomercial. In some examples, after reviewing the report, the report may be provided to the subject, either directly or indirectly (e.g., electronically or standard mail) and an optional customer service meeting/call is set up for those who desire to go over the results and product recommendations, and/or have questions regarding their report.

In some embodiments, a method of characterizing a subject's weight is provided which includes generating a personalized weight profile. In some examples, generating a personalized weight profile includes determining a subject's genetic potential in at least one area of weight health or appearance by analyzing one or more weight health-associated single nucleotide polymorphisms (SNPs) or other genetic marker associated with the particular area of weight health being assessed in a biological sample obtained from the subject. In some examples, a subject's genetic potential is determined by analyzing one or more weight health-associated SNPs or other genetic marker associated with weight health in a sample obtained from the subject. The one or more SNPs or other genetic markers associated with weight health can include one or more of obesity or weight gain or loss related, carbohydrate, fat, protein and/or alcohol or caffeine utilization or metabolism, leptin, ghrelin and hunger, satiety or cravings, blood sugar regulation and stabilization, dopamine or neurochemistry, caloric utilization and exercise SNPs or any combination thereof or any other genetic markers or categories affecting healthy weight and any combination thereof. The generated weight profile reveals the subject's genetic strengths, weaknesses and/or risks and priorities related to the one or more areas of weight health thereby allowing a personalized weight, nutritional and/or exercise regimen or nutritional/diet/food products, supplements, food alternatives (shakes, bars, snacks), behavioral or lifestyle programs, nutritional or dietary eating program or regimen recommendations and/or exercise protocols to be developed and implemented. In some examples, a disclosed method of characterizing a subject's weight further includes identifying SNPs or other genetic markers associated with a particular area of weight health. For example, SNPs associated with obesity or weight gain or loss, carbohydrate, fat, protein and/or alcohol or caffeine utilization or metabolism, leptin, ghrelin and hunger, satiety or cravings, blood sugar regulation and stabilization, dopamine or neurochemistry, caloric utilization and exercise SNPs can be identified by searching the publically available SNP on the Worldwide Web (see for example, domain name ncbi.nlm.nih.gov/snp; domain name ncbi.nlm.nih.gov/projects/SNP/; or domain name snp.cshl.org/) and determining SNPs associated with particular weight predisposition. In some examples, a disclosed method of characterizing a subject's weight further includes providing the results of the characterization study to the subject. In some examples a disclosed method of characterizing a subject's weight further includes recommending and/or providing one or more weight treatments or protocols to the subject based upon the weight profile generated by the characterization analysis. In some examples, the disclosed method of characterizing a subject's weight is performed at home. For example, a subject utilizes a kit designed to allow a subject to generate a weight profile by obtaining a DNA sample at home with the kit which includes an instruction booklet, a questionnaire, a DNA swab, a collection envelope with or without a desiccant to place the specimen in after collection. This sample is then sent for analysis and evaluation and a report is generated for the individual.

In some examples, the kit includes a means for obtaining a biological sample, such a buccal swab and a collection vial which allows the sample to be stored during shipment to the analysis laboratory. The instructions for use can be in any form, such as in a pamphlet or provided via electronic means, such as a website on the Worldwide Web.

In some examples, the disclosed method includes identifying one or more, such as two, three, four, five, or six, seven or eight or more categories of weight health and clinical studies to prove these categories have an impact on a subject's body weight. For example, one or more SNP is identified by identifying an SNP with an RS number that affects the enzyme or function in each category. Studies, such as clinical studies, are performed to identify the variations of the base at this location to validate that it is an SNP versus an infrequent variant and the clinical significance of this SNP on obesity or weight gain or loss related, carbohydrate, fat, protein and/or alcohol or caffeine utilization or metabolism, leptin, ghrelin and hunger, satiety or cravings, blood sugar regulation and stabilization, dopamine or neurochemistry, caloric utilization and exercise SNPs or any combination thereof or other SNP affects weight health Clinical research such as the GWAS are to provide information on which base patterns/SNP's are potentially protective versus risk promoting. The impact of the one or more SNPs identified on each weight category is determined, such as by use of an algorithm. Additional studies are then performed to show that the disclosed treatments/protocols impact that area of weight health.

i. Obtaining a Biological Sample

Biological samples include all clinical samples useful for generating a weight profile for a subject, including, but not limited to, cells, tissues, and bodily fluids (such as saliva); biopsied or surgically removed tissue, including tissues that are, for example, unfixed, frozen, fixed in formalin and/or embedded in paraffin; tears; skin scrapes; or surface washings. In a particular example, a sample includes cells collected by using a buccal swab or by an oral rinse.

In some examples, a sample including nucleic acids is obtained from a subject who is suspected to have or develop a genetic predisposition to a weight condition (such as diabetes or obesity), disorder or disease from bodily fluids, such as blood, urine, salvia, tears and the like. In some examples, a sample including nucleic acids is obtained from a subject who is suspected to have or develop a genetic predisposition to a weight condition (such as diabetes or obesity), disorder or disease by a buccal swab. In some examples, the subject is displaying one or more signs or symptoms of weight condition or disorder, such as undesired weight gain or weight loss, altered BMI, diabetes, hypertension, cardiovascular disease, low self-image, depression, social isolation or other signs or symptoms associated with undesired weight gain or loss. In some examples, a sample is obtained from a subject that has family members who have or have had one or more weight disorders or diseases (such as obesity, diabetes etc.). In some examples, a sample is obtained from a subject that does not show any or minimal signs or symptoms of a weight condition.

ii. Measuring Weight Health-Associated SNPs or Other Genetic Markers

The disclosed methods include measuring weight health-associated SNPs or other genetic markers in the biological sample obtained from the subject and comparing that to a control or reference value. In some examples, a subject's genetic potential is determined in eight areas of weight health or more by analyzing one or more weight health-associated SNPs associated with the eight areas or more of weight health in a sample obtained from the subject. The one or more areas of weight health can include assessing SNPs associated with obesity or weight gain or loss, carbohydrate, fat, protein and/or alcohol or caffeine utilization or metabolism, leptin, ghrelin and hunger, satiety or cravings, blood sugar regulation and stabilization, dopamine or neurochemistry, caloric utilization and exercise SNPs or any combination thereof. Each of these factors is described in detail below. In some examples, a subject's genetic potential is determined in one or more areas of weight health by measuring one or more weight health-associated SNPs with an RS number listed in Tables 1 and/or 2; each of these SNPs is incorporated herein by reference as available to the public such as on the Worldwide Web (see for example, domain name snpedia.com/index.php/; domain name ncbi.nlm.nih.gov/snp; domain name ncbi.nlm.nih.gov/projects/SNP/; domain name snp.cshl.org/) on Jan. 16, 2015.

TABLE 1
Exemplary SNPs associated with weight health.

| rs number | Gene | Gene Name | Gene Function | Category | Notes | MAF | Chromosome |
|---|---|---|---|---|---|---|---|
| rs11724758 | FABP2 | Fatty Acid | Glucoregulatory | Fat | Influences levels | 0.4674 | 4q28-q31 |
| | | Binding | function and lipid | | of blood lipids | | |
| | | Protein 2 | oxidation, fat | | | | |
| | | | absorption in | | | | |
| | | | the gut | | | | |
| rs11724320 | NPY1R | Neuroopeptide | Receptor for | Fat/carbs | Influences intake | 0.47 | 4q31.3-q32 |
| | | Y 1 Receptor | neuropeptide Y | | of mono and | | |
| | | | and peptide YY | | disaccharides | | |
| rs2272382 | TUB | Tubby | Role in obesity | Fats/carbs | Assoc. with body | 0.3687 | 11p15.5 |
| | | bitartite | | | composition and | | |
| | | transcription | | | eating behavior; | | |
| | | factor | | | more central | | |
| | | | | | obesity after | | |
| | | | | | menopause | | |
| rs553668 | ADRA2A | Alpha 2 | Regulation of | Fats/carbs | Associated with | 0.2902 | 10q25.2 |
| | | Andrenergic | insulin secretion | | T2DM in people | | |
| | | Receptor | and lipolysis | | with BMI <25 | | |
| rs894160 | PLIN | Perilipin | cAMP dependent | Fats/carbs | Increase in type 2 | 0.3324 | 15q26 |
| | | | protein kinase | | diabetes risk in | | |
| | | | substrate. | | women without | | |
| | | | Costs lipid | | central obesity | | |
| | | | droplets and | | | | |
| | | | involved in | | | | |
| | | | regulation of | | | | |
| | | | lipolysis | | | | |
| rs1042714 | ADRB2 | Adrenoceptor | Interacts with | Fats | Influences risk | 0.2043 | 5q31-q32 |
| | | beta 2 | class C L-type | | of type 2 diabetes | | |
| | | | calcium channel | | | | |
| rs9939609 | FTO | Fat Mass and | Non heme iron | Carbs/hunger | Positive (increased | 0.3554 | 16q12.2 |
| | | Obesity | enzyme; hunger | and satiety | weight) impact on | | |
| | | Associated | and satiety | | BMI; increase in | | |
| | | | | | fat mass; nutrient | | |
| | | | | | sensing, mRNA | | |
| | | | | | translation; | | |
| | | | | | growth | | |
| rs12255372 | TCFL2 | Transcription | Involved in Wnt | Carbs | Associated with | 0.2029 | 10q25.3 |
| | | factor 7 like 2 | signaling; | | type 2 diabetes | | |
| | | | glucose | | | | |
| | | | homeostasis | | | | |
| rs182549 | MCM6 | Minichromosome | Necessary | Carbs | C allele | 0.2332 | 2q21 |
| | | maintenance | for genome | | associated with | | |
| | | complex | replication | | lactose | | |
| | | component 6 | | | intolerance | | |
| rs7072268 | HK1 | Hexokinase 1 | Involved in | Carbs | Associated with | 0.4862 | 10q22 |
| | | | glucose | | congenital | | |
| | | | metabolism | | hyperinsulinemia | | |
| rs13266634 | SLC30A8 | Solute carrier 30 | Involved in | Carbs | Loss of function | 0.2824 | 18q24.11 |
| | | | insulin | | protects against | | |
| | | | secretion | | T2DM | | |
| rs1402837 | G6PC2 | Glucose-6 | Involved in | Carbs | Influences | 0.2888 | 2q24.3 |
| | | phosphatase | release of | | hemoglobin | | |
| | | catalytic 2 | glucose into | | A1C levels | | |
| | | | bloodstream | | | | |
| rs4680 | COMT | Catechol-O- | Transfers | Neuro- | Breakdown of | 0.3692 | 22q11.21 |
| | | Methyltransferase | methyl group | chemistry | catecholamines | | |
| | | | from SAMe to | | | | |
| | | | catecholamines | | | | |
| rs1800497 | DRD2 | Dopamine | Binds | Neuro- | Influences number | 0.2961 | 11q23 |
| | | Receptor | dopamine | chemistry | of dopamine | | |
| | | D2 | | | binding sites | | |
| | | | | | in brain | | |
| rs1137101 | LEPR | Leptin Receptor | Binds leptin; | Hunger and | Helps maintain | 0.4104 | 1p31 |
| | | | involved in | Satiety | healthy levels | | |
| | | | glucose | | of blood sugar | | |
| | | | homeostasis | | | | |
| rs5082 | APOA2 | Apolipoprotein | Second most | Hunger and | Greater weight | 0.2521 | 1q23.3 |
| | | A2 | abundant | Satiety/Fats | gain in response | | |
| | | | protein in | | to fat intake; | | |
| | | | HDL particles | | modulates | | |
| | | | | | ghrelin | | |
| rs762551 | CYP1A2 | Cytochrome | Caffeine | Caffeine | decreased | 0.3522 | 15q24.1 |
| | | P-450 1A2 | metabolism | | activity of | | |
| | | | and detox | | enzyme; | | |

TABLE 2
Exemplary SNPs associated with weight health.

rs1137101
rs662799
rs7903146
rs2272383
rs1528133
rs3820710
rs1801282
rs2232165
rs193922341
rs730497
rs11724758
rs1801133
rs1800872
rs11724320
rs16944
rs1042031
rs231775
rs2272382
rs1799883
rs4961
rs1042714
rs553668
rs1800629
rs5370
rs894160
rs6296
rs1800795
rs9939609
rs1800796
rs7493
rs213950
rs12255372
rs1799983
rs328
rs268
rs182549
rs2383206
rs1800861
rs7072268
rs1801253
rs2227564
rs13266634
rs731236
rs1544410
rs7975232
rs6313
rs1402837
rs2236225
rs1800588
rs4680
rs243865
rs4673
rs708272
rs1800497
rs4291
rs4343
rs429358
rs5082
rs762551
rs7412
rs1800469
rs601338
rs688
rs7121
rs234706
rs4680

Methods of isolating nucleic acid molecules from a biological sample are routine and known to those of ordinary skill in the art, for example using PCR to amplify the molecules from the sample, or by using a commercially available kit to isolate DNA. Nucleic acid molecules isolated from buccal swab samples or any other biological sample can be amplified using routine methods to form nucleic acid amplification products. Exemplary methods of isolating DNA and detecting SNPs associated with one or more weight conditions or disorders are described below in the Molecular Methods Section.

iii. Providing a Weight Profile to a Subject

Following the measurement of one or more SNPs or other genetic markers associated with weight health, the results, findings, diagnoses, predictions and/or treatment recommendations can be provided to the subject. For example, the results, findings, diagnoses, predictions and/or treatment recommendations can be recorded and communicated to technicians, physicians and/or patients or clients. In certain embodiments, computers will be used to communicate such information to interested parties, such as, clients, patients and/or the attending physicians. Based on the measurement, the therapy or protocol administered to a subject can be started, modified not started or re-started (in the case of monitoring for a reoccurrence of a particular weight condition/disorder).

It is contemplated that the disclosed weight profile can be used in both clinical and non-clinical settings. In particular, the disclosed methods and assays are capable of assisting individuals who are interested in altering their weight or maintaining their weight. In some examples, the individual does not display any or minimal signs or symptoms of a weight disorder/condition/disease. In some examples, the individual desires to alter their weight, but are not clinically obese. In some examples, individuals interested in altering their weight can obtain a weight profile and then based upon that be provided recommendations as to nutritional/diet/food products, supplements, food alternatives (shakes, bars, snacks), behavioral or lifestyle programs, nutritional or dietary eating program or regimen recommendations and/or exercise regimens they should follow. These recommendations can then be provided either in verbal or written communication. In some examples, the recommendations are provided to the individual via a computer or in written format and accompany the weight profile. For example, a subject may request their weight profile and suggested nutritional/diet/food products, supplements, food alternatives (shakes, bars, snacks), behavioral or lifestyle programs, nutritional Or dietary eating program or regimen recommendations and/or exercise regimens be provided to them via electronic means, such as by email. In some examples, the recommendations are provided in person, such as by a healthcare provider, nutritionist or a non-clinical provider. In other examples, the recommendations are provided by a physician. In other examples recommendations are made via computer, email, the internet.

In some examples, the output can provide a recommended therapeutic regimen or weight maintenance protocol including nutritional/diet/food products, supplements, food alternatives (shakes, bars, snacks), behavioral Or lifestyle programs, nutritional or dietary eating program Or regimen recommendations and/or exercise regimens. In some examples, the test may include determination of other clinical or non-clinical information.

In some embodiments, the disclosed methods include one or more of the following depending on the subject's weight profile: a) prescribing or recommending a protocol or treatment regimen for the subject if the subject's determined profile is considered to be sub-optimal or deficient in one or more areas of weight health; b) not prescribing or recommending a protocol or treatment regimen for the subject if the subject's determined weight profile is considered to be optimal in the evaluated weight areas; c) administering a protocol or treatment to the subject if the subject's determined diagnosis or profile is considered to be sub-optimal or deficient in one or more areas of weight health; d) not administering a protocol or treatment regimen to the subject if the subject's determined weight profile is considered to be optimal in the evaluated weight areas. In an alternative embodiment, the method can include recommending one or more of a)-d).

In one embodiment, a diagnosis, prediction and/or treatment recommendation or protocol based on the weight profile disclosed herein is communicated to the subject as soon as possible after the assay is completed and the diagnosis and/or prediction is generated. The results and/or related information may be communicated to the subject by the subject's treating physician, nutritionist, nurse, nurse practitioner or non-clinical advisor, such as a weight loss coach or other qualified person. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a weight profile analysis and/or conclusions drawn from and/or treatment recommendations or protocols based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments of the methods of the disclosure, all or some of the method steps, including the assaying of samples, performing the comparisons, and/or communicating of assay results, diagnoses or recommendations, may be carried out in diverse (e.g., foreign) jurisdictions.

In several embodiments, identification of a subject as having or at risk of developing a weight condition or disorder results in the physician treating the subject, such as prescribing one or more therapeutic agents for inhibiting or delaying one or more signs and symptoms associated with the disorder/condition. In additional embodiments, the treatment, dose or dosing regimen is modified based on the information obtained using the methods disclosed herein.

The subject can be monitored while undergoing treatment using the methods described herein in order to assess the efficacy of the treatment or protocol. In this manner, the length of time or the amount given to the subject can be modified based on the results obtained using the methods disclosed herein. The subject can also be monitored after the treatment using the methods described herein to monitor for relapse and thus, the effectiveness of the given treatment. In this manner, whether to resume treatment can be decided based on the results obtained using the methods disclosed herein. In some examples, this monitoring is performed by a clinical healthcare provider. In other examples, this monitoring is performed by a non-clinical provider and can include self-monitoring or monitoring by a weight consultant.

In some embodiments, once a subject's weight profile is determined, an indication of that profile can be displayed and/or conveyed to a clinician, caregiver or a non-clinical provider, including the client/subject. For example, the results of the test are provided to a user (such as a clinician or other health care worker, laboratory personnel, or patient) in a perceivable output that provides information about the results of the test. In some examples, the output is a paper output (for example, a written or printed output), a display on a screen, a graphical output (for example, a graph, chart, or other diagram), or an audible output.

In other examples, the output is a numerical value, such as an amount of a particular set of SNPs or other genetic markers in the sample as compared to a control. In additional examples, the output is a graphical representation, for example, a graph that indicates the value (such as amount or relative amount) of the set of SNPs or other genetic markers in the sample from the subject on a standard curve. In a particular example, the output (such as a graphical output) shows or provides a cut-off value or level that indicates the presence of optimal, sub-optimal or deficient weight factor level. In some examples, the output is communicated to the user, for example by providing an output via physical, audible, or electronic means (for example by mail, telephone, facsimile transmission, email, or communication to an electronic medical record).

The output can provide quantitative information (for example, an amount of an molecule in a test sample compared to a control sample or value) or can provide qualitative information (for example, detection of an alteration in hunger and cravings related to leptin sensitivity and function may alter food recommendation, exercise recommendations and supplement recommendation to support healthy leptin sensitivity and function and/or deficiency in ghrelin for healthy physiologic hunger and cravings for healthy weight). In additional examples, the output can provide qualitative information regarding the relative amount of a particular SNP or other genetic marker in the sample, such as identifying presence of an increase relative to a control, a decrease relative to a control, or no change relative to a control.

In some examples, the output is accompanied by guidelines for interpreting the data, for example, numerical or other limits that indicate the presence or absence of a weight disorder/condition/priority/risk. The indicia in the output can, for example, include normal or abnormal ranges or a cutoff, normal, subnormal or deficient ratings which the recipient of the output may then use to interpret the results, for example, to arrive at a diagnosis, prognosis, susceptibility towards or treatment plan.

iv. Providing Weight Treatment/Protocol to a Subject

In some embodiments, the method further includes providing an appropriate therapy or protocol for the subject after reviewing the weight profile. For example, a subject diagnosed with a weight disorder/condition can be provided a particular therapy including nutritional/diet/food products, supplements, food alternatives (shakes, bars, snacks), behavioral Of lifestyle programs, nutritional or dietary eating program or regimen recommendations and/or exercise regimens. In some examples, the therapy includes administering an agent to alter one or more signs or symptoms associated with the identified weight disorder/condition. In some examples, one or more of the agents described below are administered to a subject to treat or support one or more sub-normal or deficient weight factors identified by the disclosed weight profile. In some examples, providing weight treatment includes providing a dietary and/or exercise or lifestyle regimen. For example, suggestions regarding diet can be given wherein a subject is provided a protocol of particular foods, supplements, food alternatives (such as shakes, bars, snacks and the like) to consume and/or avoid. The treatment/protocol can be performed multiple times for optimal results. In one embodiment, the treatment is performed twice a day. In another embodiment, the treatment is performed daily. In other embodiments, the recommendation/treatment is performed weekly. In another embodiment, the treatment is performed monthly. In another embodiment, the treatment is performed at least once every one to two days. In another embodiment, the treatment is performed at least once every one to two weeks.

It is contemplated that the desired treatments or protocols may be administered via any means known to one of skill in the art, including oral administration. In some examples, a composition is administered to the subject orally, such as in a capsule or tablet. It is contemplated that one or more compositions can be administered via multiple routes as the same or different time period depending upon the disorders/conditions being treated. In some examples, the composition is provided in a powder beverage mix, liquid beverage, a snack bar, or other food product. In some examples, a composition is provided with changes in dietary recommendations along with supplements to support health cellular function. In some examples, the treatment includes dietary restrictions, administration of one or more nutraceuticals/supplements, and an exercise or lifestyle regimen. The percentage of improvement can be, for example, at least about a 5%, such as at least about 10%, at least a 15%, at least a 20%, at least about 30%, at least about 40%, at least about 50%, at least about 60%, at least about 70%, at least about 80%, at least about 90% or at least about 100% change compared to the baseline score prior to treatment with one or more weight altering/controlling agents including those described herein. The improvement can be measured by both subjective and objective methods, and can be quantified using a subjective scoring or a panel scoring, amongst other methods. “Baseline” is the score prior to treatment and can include measurements such as BMI, electrical impedance, dexa scanning, or any other quantitative measurement such as simply change is clothing size or measurements.

### Molecular Methods

Generally, the methods disclosed herein involve an assessment of nucleic acid sequence. Molecular techniques of use in all of these methods are disclosed below.

Preparation of Nucleic Acids for Analysis:

Nucleic acid molecules can be prepared for analysis using any technique known to those skilled in the art. Generally, such techniques result in the production of a nucleic acid molecule sufficiently pure to determine the presence or absence of one or more variations at one or more locations in the nucleic acid molecule. Such techniques are described for example, in Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), incorporated herein by reference.

When the nucleic acid of interest is present in a cell, it can be necessary to first prepare an extract of the cell and then perform further steps, such as differential precipitation, column chromatography, extraction with organic solvents and the like, in order to obtain a sufficiently pure preparation of nucleic acid. Extracts can be prepared using standard techniques in the art, for example, by chemical or mechanical lysis of the cell. Extracts then can be further treated, for example, by filtration and/or centrifugation and/or with chaotropic salts such as guanidinium isothiocyanate or urea or with organic solvents such as phenol and/or HCCl₃ to denature any contaminating and potentially interfering proteins. When chaotropic salts are used, it can be desirable to remove the salts from the nucleic acid-containing sample. This can be accomplished using standard techniques in the art such as precipitation, filtration, size exclusion chromatography and the like.

In some instances, messenger RNA can be extracted from cells. Techniques and material for this purpose are known to those skilled in the art and can involve the use of oligo dT attached to a solid support such as a bead or plastic surface. In some embodiments, the mRNA can be reversed transcribed into cDNA using, for example, a reverse transcriptase enzyme. Suitable enzymes are commercially available from, for example, Invitrogen, Carlsbad Calif. Optionally, cDNA prepared from mRNA can also be amplified.

Amplification of Nucleic Acid Molecules:

Optionally, the nucleic acid samples obtained from the subject are amplified prior to detection. Target nucleic acids are amplified to obtain amplification products, including sequences from a haplotype block including a tag SNP, can be amplified from the sample prior to detection. Typically, DNA sequences are amplified, although in some instances RNA sequences can be amplified or converted into cDNA, such as by using RT PCR.

Any nucleic acid amplification method can be used. An example of in vitro amplification is the polymerase chain reaction (PCR), in which a biological sample obtained from a subject is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of in vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see PCT Publication NO. WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

In specific examples, the target sequences to be amplified from the subject include a nucleotide sequence of interest including the SNP. In an embodiment, a single SNP with exceptionally high predictive value is amplified, or a nucleic acid encoding the SNP is amplified.

A pair of primers can be utilized in the amplification reaction. One or both of the primers can be labeled, for example with a detectable radiolabel, fluorophore, or biotin molecule. The pair of primers includes an upstream primer (which binds 5′ to the downstream primer) and a downstream primer (which binds 3′ to the upstream primer). The pair of primers used in the amplification reactions are selective primers which permit amplification of a size related marker locus. Numerous primers can be designed by those of skill in the art simply by determining the sequence of the desired target region, for example, using well known computer assisted algorithms that select primers within desired parameters suitable for annealing and amplification.

If desired, an additional pair of primers can be included in the amplification reaction as an internal control. For example, these primers can be used to amplify a “housekeeping” nucleic acid molecule, and serve to provide confirmation of appropriate amplification. In another example, a target nucleic acid molecule including primer hybridization sites can be constructed and included in the amplification reactor. One of skill in the art will readily be able to identify primer pairs to serve as internal control primers.

Primer Design Strategy:

Increased use of polymerase chain reaction (PCR) methods has stimulated the development of many programs to aid in the design or selection of oligonucleotides used as primers for PCR. Four examples of such programs that are freely available via the Internet are: PRIMER™ by Mark Daly and Steve Lincoln of the Whitehead Institute (UNIX, VMS, DOS, and Macintosh), Oligonucleotide Selection Program by Phil Green and LaDeana Hiller of Washington University in St. Louis (UNIX, VMS, DOS, and Macintosh), PGEN™ by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

Designing oligonucleotides for use as either sequencing or PCR primers to detect requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding programs. If a possible stem structure is observed, the sequence of the primer can be shifted a few nucleotides in either direction to minimize the predicted secondary structure. When the amplified sequence is intended for subsequence cloning, the sequence of the oligonucleotide can also be compared with the sequences of both strands of the appropriate vector and insert DNA. A sequencing primer only has a single match to the target DNA. It is also advisable to exclude primers that have only a single mismatch with an undesired target DNA sequence. For PCR primers used to amplify genomic DNA, the primer sequence can be compared to the sequences in the GENBANK™ database to determine if any significant matches occur. If the oligonucleotide sequence is present in any known DNA sequence or, more importantly, in any known repetitive elements, the primer sequence should be changed.

Detection of Alleles:

The nucleic acids obtained from the sample can be genotyped to identify the particular allele present for a marker locus. A sample of sufficient quantity to permit direct detection of marker alleles from the sample can be obtained from the subject. Alternatively, a smaller sample is obtained from the subject and the nucleic acids are amplified prior to detection. Any target nucleic acid that is informative for a chromosome haplotype can be detected. Any method of detecting a nucleic acid molecule can be used, such as hybridization and/or sequencing assays.

Hybridization is the binding of complementary strands of DNA, DNA/RNA, or RNA. Hybridization can occur when primers or probes bind to target sequences such as target sequences within genomic DNA. Probes and primers that are useful generally include nucleic acid sequences that hybridize (for example under high stringency conditions) with a nucleic acid sequence including the SNP of interest, but do not hybridize to a reference allele, or that hybridize to the reference allele, but do not hybridize to the SNP. Physical methods of detecting hybridization or binding of complementary strands of nucleic acid molecules, include but are not limited to, such methods as DNase I or chemical footprinting, gel shift and affinity cleavage assays, Southern and Northern blotting, dot blotting and light absorption detection procedures. The binding between a nucleic acid primer or probe and its target nucleic acid is frequently characterized by the temperature (Tₘ) at which 50% of the nucleic acid probe is melted from its target. A higher (Tₘ) means a stronger or more stable complex relative to a complex with a lower (Tm).

Generally, complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence (such as an SNP associated with a particular weight factor) to achieve detectable and specific binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity. The qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning: a laboratory manual, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Complementarity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share at Least 80% Complementarity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share at Least 50% Complementarity)

Hybridization: 6×SSC at RT to 55° C. for 16-20 hours

Wash at least twice: 2×-3×SSC at RT to 55° C. for 20-30 minutes each.

Methods for labeling nucleic acid molecules so they can be detected are well known. Examples of such labels include non-radiolabels and radiolabels. Non-radiolabels include, but are not limited to an enzyme, chemiluminescent compound, fluorescent compound (such as FITC, Cy3, and Cy5), metal complex, hapten, enzyme, colorimetric agent, a dye, or combinations thereof. Radiolabels include, but are not limited to, ¹²⁵I, ³²P and ³⁵S. For example, radioactive and fluorescent labeling methods, as well as other methods known in the art, are suitable for use with the present disclosure. In one example, primers used to amplify the subject's nucleic acids are labeled (such as with biotin, a radiolabel, or a fluorophore). In another example, amplified target nucleic acid samples are end-labeled to form labeled amplified material. For example, amplified nucleic acid molecules can be labeled by including labeled nucleotides in the amplification reactions.

Nucleic acid molecules corresponding to one or more tag SNPs or haplotype blocks including the tag SNP can also be detected by hybridization procedures using a labeled nucleic acid probe, such as a probe that detects only one alternative allele at a marker locus. Most commonly, the target nucleic acid (or amplified target nucleic acid) is separated based on size or charge and transferred to a solid support. The solid support (such as membrane made of nylon or nitrocellulose) is contacted with a labeled nucleic acid probe, which hybridizes to it complementary target under suitable hybridization conditions to form a hybridization complex.

Hybridization conditions for a given combination of array and target material can be optimized routinely in an empirical manner close to the Tₘ of the expected duplexes, thereby maximizing the discriminating power of the method. For example, the hybridization conditions can be selected to permit discrimination between matched and mismatched oligonucleotides. Hybridization conditions can be chosen to correspond to those known to be suitable in standard procedures for hybridization to filters (and optionally for hybridization to arrays). In particular, temperature is controlled to substantially eliminate formation of duplexes between sequences other than an exactly complementary allele of the selected marker. A variety of known hybridization solvents can be employed, the choice being dependent on considerations known to one of skill in the art (see U.S. Pat. No. 5,981,185).

Once the target nucleic acid molecules have been hybridized with the labeled probes, the presence of the hybridization complex can be analyzed, for example by detecting the complexes.

Methods for detecting hybridized nucleic acid complexes are well known in the art. In one example, detection includes detecting one or more labels present on the oligonucleotides, the target (e.g., amplified) sequences, or both. Detection can include treating the hybridized complex with a buffer and/or a conjugating solution to effect conjugation or coupling of the hybridized complex with the detection label, and treating the conjugated, hybridized complex with a detection reagent. In one example, the conjugating solution includes streptavidin alkaline phosphatase, avidin alkaline phosphatase, or horseradish peroxidase. Specific, non-limiting examples of conjugating solutions include streptavidin alkaline phosphatase, avidin alkaline phosphatase, or horseradish peroxidase. The conjugated, hybridized complex can be treated with a detection reagent. In one example, the detection reagent includes enzyme-labeled fluorescence reagents or calorimetric reagents. In one specific non-limiting example, the detection reagent is enzyme-labeled fluorescence reagent (ELF) from Molecular Probes, Inc. (Eugene, Oreg.). The hybridized complex can then be placed on a detection device, such as an ultraviolet (UV) transilluminator (manufactured by UVP, Inc. of Upland, Calif.). The signal is developed and the increased signal intensity can be recorded with a recording device, such as a charge coupled device (CCD) camera (manufactured by Photometrics, Inc. of Tucson, Ariz.). In particular examples, these steps are not performed when radiolabels are used. In particular examples, the method further includes quantification, for instance by determining the amount of hybridization.

Allele Specific PCR:

Allele-specific PCR differentiates between target regions differing in the presence of absence of a variation or polymorphism. PCR amplification primers are chosen based upon their complementarity to the target sequence, such as nucleic acid sequence in including a SNP, a specified region of an allele including an SNP, or to the SNP itself. The primers bind only to certain alleles of the target sequence. This method is described by Gibbs, Nucleic Acid Res. 17:12427 2448, 1989, herein incorporated by reference. In some examples, a commercially available assay based upon allele-specific PCR SNP detection chemistry is utilized, such as a SNPtype™ Assay, Dynamic Array or other SNP genotyping product from FUJEDIGM® is utilized.

Allele Specific Oligonucleotide Screening Methods:

Further screening methods employ the allele-specific oligonucleotide (ASO) screening methods (e.g. see Saiki et al., Nature 324:163-166, 1986). Oligonucleotides with one or more base pair mismatches are generated for any particular allele or haplotype block. ASO screening methods detect mismatches between one allele (or haplotype block) in the target genomic or PCR amplified DNA and the other allele (or haplotype block), showing decreased binding of the oligonucleotide relative to the second allele (i.e. the other allele) oligonucleotide. Oligonucleotide probes can be designed that under low stringency will bind to both polymorphic forms of the allele, but which at high stringency, only bind to the allele to which they correspond. Alternatively, stringency conditions can be devised in which an essentially binary response is obtained, i.e., an ASO corresponding to a variant form of the target gene will hybridize to that allele (haplotype block), and not to the reference allele (haplotype block).

Ligase Mediated Allele Detection Method:

Ligase can also be used to detect point mutations in a ligation amplification reaction (e.g. as described in Wu et al., Genomics 4:560-569, 1989). The ligation amplification reaction (LAR) utilizes amplification of specific DNA sequence using sequential rounds of template dependent ligation (e.g. as described in Wu, supra, and Barany, Proc. Nat. Acad. Sci. 88:189-193, 1990).

Denaturing Gradient Gel Electrophoresis:

Amplification products generated using the polymerase chain reaction can be analyzed by the use of denaturing gradient gel electrophoresis. Different alleles (haplotype blocks) can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (T_{M}). Melting domains are at least 20 base pairs in length, and can be up to several hundred base pairs in length.

Differentiation between alleles (haplotype blocks) based on sequence specific melting domain differences can be assessed using polyacrylamide gel electrophoresis, as described in Chapter 7 of Erlich, ed., PCR Technology, Principles and Applications for DNA Amplification, W. H. Freeman and Co., New York (1992).

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527, 1986, and Myers et al., in Genomic Analysis, A Practical Approach, K. Davies Ed. IRL Press Limited, Oxford, pp. 95 139, 1988. The electrophoresis system is maintained at a temperature slightly below the Tm of the melting domains of the target sequences.

In an alternative method of denaturing gradient gel electrophoresis, the target sequences can be initially attached to a stretch of GC nucleotides, termed a GC clamp, as described in Chapter 7 of Erlich, supra. In one example, at least 80% of the nucleotides in the GC clamp are either guanine or cytosine. In another example, the GC clamp is at least 30 bases long. This method is particularly suited to target sequences with high Tₘ s.

Generally, the target region is amplified by polymerase chain reaction. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions. DNA fragments differing by a single base change will migrate through the gel to different positions, which can be visualized by ethidium bromide staining.

Temperature Gradient Gel Electrophoresis:

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis:

Target sequences, such as alleles or haplotype blocks can be differentiated using single-strand conformation polymorphism analysis, which identifies base differences by alteration in electrophoretic migration of single stranded PCR products, for example as described in Orita et al., Proc. Nat. Acad. Sci. 85:2766-2770, 1989. Amplified PCR products can be generated as described above, and heated or otherwise denatured, to form single stranded amplification products. Single-stranded nucleic acids can refold or form secondary structures which are partially dependent on the base sequence. Thus, electrophoretic mobility of single-stranded amplification products can detect base-sequence difference between alleles or haplotype blocks.

Chemical or Enzymatic Cleavage of Mismatches:

Differences between target sequences, such as alleles or haplotype blocks, can also be detected by differential chemical cleavage of mismatched base pairs, for example as described in Grompe et al., Am. J. Hum. Genet. 48:212-222, 1991. In another method, differences between target sequences, such as alleles or haplotype blocks, can be detected by enzymatic cleavage of mismatched base pairs, as described in Nelson et al., Nature Genetics 4:11-18, 1993. Briefly, genetic material from an animal and an affected family member can be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms.

Non-Gel Systems:

Other possible techniques include non-gel systems such as TaqMan™ (Perkin Elmer). In this system oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete (there is a mismatch of some form) the cleavage of the dye does not take place. Thus only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Non-PCR Based Allele Detection:

The identification of a DNA sequence can be made without an amplification step, based on polymorphisms including restriction fragment length polymorphisms in a subject and a control, such as a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes can be labeled directly or indirectly, such that by assaying for the presence or absence of the probe, one can detect the presence or absence of the target sequence. Direct labeling methods include radioisotope labeling, such as with ³²P or ³⁵S. Indirect labeling methods include fluorescent tags, biotin complexes which can be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to a nucleic acid sequence wherein a polymorphism is present that is associated with a particular weight factor, and thus defining a genetic marker, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Exemplary tandem repeat hybridization probes for use in the methods disclosed are those that recognize a small number of fragments at a specific locus at high stringency hybridization conditions, or that recognize a larger number of fragments at that locus when the stringency conditions are lowered.

Arrays for Detecting Nucleic Acid:

In particular examples, the methods can be performed using an array that includes a plurality of markers. Such arrays can include nucleic acid molecules. In one example, the array includes nucleic acid oligonucleotide probes that can hybridize to one or more alleles.

Arrays can be used to detect the presence of amplified sequences including one or more SNPs of interest using specific oligonucleotide probes. Additionally, if an internal control nucleic acid sequence was amplified in the amplification reaction, an oligonucleotide probe can be included to detect the presence of this amplified nucleic acid molecule. The oligonucleotide probes bound to the array can specifically bind sequences amplified in the amplification reaction (such as under high stringency conditions).

The methods and apparatus in accordance with the present disclosure takes advantage of the fact that under appropriate conditions oligonucleotides form base-paired duplexes with nucleic acid molecules that have a complementary base sequence. The stability of the duplex is dependent on a number of factors, including the length of the oligonucleotides, the base composition, and the composition of the solution in which hybridization is performed. The effects of base composition on duplex stability can be reduced by carrying out the hybridization in particular solutions, for example in the presence of high concentrations of tertiary or quaternary amines.

The thermal stability of the duplex is also dependent on the degree of sequence similarity between the sequences. By carrying out the hybridization at temperatures close to the anticipated Tₘ's of the type of duplexes expected to be formed between the target sequences and the oligonucleotides bound to the array, the rate of formation of mis-matched duplexes can be substantially reduced.

The length of each oligonucleotide sequence employed in the array can be selected to optimize binding to a specific allele of a marker locus associated with a particular weight factor. An optimum length for use with a particular marker nucleic acid sequence under specific screening conditions can be determined empirically. Thus, the length for each individual element of the set of oligonucleotide sequences included in the array can be optimized for screening. In one example, oligonucleotide probes are from about 20 to about 35 nucleotides in length or about 25 to about 40 nucleotides in length.

The oligonucleotide probe sequences forming the array can be directly linked to the support, for example via the 5′- or 3′-end of the probe. In one example, the oligonucleotides are bound to the solid support by the 5′ end. However, one of skill in the art can determine whether the use of the 3′ end or the 5′ end of the oligonucleotide is suitable for bonding to the solid support. In general, the internal complementarity of an oligonucleotide probe in the region of the 3′ end and the 5′ end determines binding to the support. Alternatively, the oligonucleotide probes can be attached to the support by sequences such as oligonucleotides or other molecules that serve as spacers or linkers to the solid support.

In particular examples, the array is a microarray formed from glass (silicon dioxide). Suitable silicon dioxide types for the solid support include, but are not limited to: aluminosilicate, borosilicate, silica, soda lime, zinc titania and fused silica (for example see Schena, Micraoarray Analysis. John Wiley & Sons, Inc, Hoboken, N.J., 2003). The attachment of nucleic acids to the surface of the glass can be achieved by methods known in the art, for example by surface treatments that form from an organic polymer. Particular examples include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof (see U.S. Pat. No. 5,985,567), organosilane compounds that provide chemically active amine or aldehyde groups, epoxy or polylysine treatment of the microarray. Another example of a solid support surface is polypropylene.

In general, suitable characteristics of the material that can be used to form the solid support surface include: being amenable to surface activation such that upon activation, the surface of the support is capable of covalently attaching a biomolecule such as an oligonucleotide thereto; amenability to “in situ” synthesis of biomolecules; being chemically inert such that at the areas on the support not occupied by the oligonucleotides are not amenable to non-specific binding, or when non-specific binding occurs, such materials can be readily removed from the surface without removing the oligonucleotides.

In one example, the surface treatment is amine-containing silane derivatives. Attachment of nucleic acids to an amine surface occurs via interactions between negatively charged phosphate groups on the DNA backbone and positively charged amino groups (Schena, Micraoarray Analysis. John Wiley & Sons, Inc, Hoboken, N.J., 2003). In another example, reactive aldehyde groups are used as surface treatment. Attachment to the aldehyde surface is achieved by the addition of 5′-amine group or amino linker to the DNA of interest. Binding occurs when the nonbonding electron pair on the amine linker acts as a nucleophile that attacks the electropositive carbon atom of the aldehyde group.

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. Biaxially oriented polypropylene (BOPP) films are also suitable in this regard; in addition to their durability, BOPP films exhibit a low background fluorescence. In a particular example, the array is a solid phase, Allele-Specific Oligonucleotides (ASO) based nucleic acid array.

The array formats of the present disclosure can be included in a variety of different types of formats. A “format” includes any format to which the solid support can be affixed, such as microtiter plates, test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as see PCT Publication No. WO 85/01051 and PCT Publication No. WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second (2°) set of rows that are now perpendicular to the first set. This process creates a multiple-channel array whose intersection generates a plurality of discrete cells.

In particular examples, the oligonucleotide probes on the array include one or more labels, which permit detection of oligonucleotide probe target sequence hybridization complexes.

Without further elaboration, it is believed that one skilled in the art can, using this description, utilize the present disclosure to its fullest extent. The following Examples are illustrative only, and not limiting of the remainder of the disclosure in any way whatsoever.

### EXAMPLES

### Example 1

This examples details a study conducted to evaluate the presently disclosed methods and protocols for effective weight loss. With use of the SNP analysis disclosed herein the genetic strength and weaknesses of the individual were utilized to make more personalized recommendations leading to a 5% or greater loss in body fat and the ability to maintain this weight loss over one year. Analysis of the data from this study confirmed the ability of the laboratory to accurately and reproducibly identify selected SNP's and verify their frequency in the population studied. Population frequencies were compared to the medical literature for accuracy. An algorithm was created to mathematically calculate the impact of each SNP on the applicable function. This example demonstrates that the disclosed methods provide clients/patients a highly personalized method by which they can objectively identify biological strengths and weaknesses in their genetic coding that affected weight gain or loss which can be used to provide effective regimens/protocols for weight control, including long term weight control.

The disclosed methods of weight analysis were utilized on clients that had previously attempted to lose weight but only had minimal results (1-2% change in body fat loss by bioimpedence) over a 12-week period of time and experienced the inability to maintain their weight loss long term—over a period of at least a year. A SNP panel was obtained on the client to determine variations in their genetic coding that can influence healthy weight and ability to lose weight. One area of focus on the healthy weight panel is hunger and satiety. Specifically, SNP's related to leptin and ghrelin sensitivity including rs1137101, rs9939609, and rs5082 and their influence on hunger, satiety, cravings and healthy weight were analyzed. Individuals with non-ideal coding were identified as high priority or high risk in this category and nutritional/diet/food products, supplements, food alternatives (shakes, bars, snacks), behavioral or lifestyle programs, nutritional or dietary eating program or regimen recommendations and/or exercise regimens were implemented to address hunger, cravings and satiety for better weight management. For example, an Eating/lifestyle Program such as the following was recommended including foods, snacks, bars, shakes, lifestyle recommendations:

Breakfast: 4 oz. assorted cheeses with sliced cucumber; 2 Omega-3 egg omelet, ½ cup swiss cheese and chopped red, yellow, and green peppers, 2 tsp. olive oil; 2 soft boiled eggs, 2 turkey sausages, or 1 slice canadian bacon, green tea—3 cups daily; 4 oz. slices lean ham with 2 oz. slices melted cheese, 2 tomato slices; 4 oz. smoked salmon, 2 Tbsp. cream cheese, 2 Wasa crackers, cucumber slices; ½ cup cottage cheese, 2 slices low sodium ham, herb tea; 2 oz. Lox, neufchatel cheese on celery stick, herb tea; or Protein Shakes with Flaxseeds
Lunch and Dinner: Buffalo or turkey burger with lettuce, tomato and cole slaw, Ketchup, mustard and pickle relish may be used if desired (all Sugar free); Season sardines in water (green and white label), green Caesar salad with or without chicken breast; marinated broiled flank steak with sautéed oyster mushrooms, roasted vegetables, green salad with flax oil dressing; large mixed green salad w/oil and lemon juice, small can of tuna, chopped yellow and sweet red pepper; chicken salad made with sugar-free Canola Oil mayonnaise, roasted vegetables, spinach salad; 4 ounces wood-smoked or broiled salmon, tomato sauce w/extra oregano, thyme, and garlic, grilled vegetables; 1 chicken breast with rosemary, vegetables with roasted onions or garlic, spinach salad with goat cheese, walnuts and balsamic vinegar/walnut oil dressing; salmon burger patties made with 6 oz. chopped salmon, onions, dill, an egg, and ¼ cup ground sesame seeds, and sautéed in skillet with 1 Tbsp. butter; gourmet salmon salad made with 1 can salmon, 2 tsp. Sliced scallions, 1 tsp. Sliced radishes, 2 tsp. Rice vinegar, 1 tsp. Flax oil, 1 tsp. Soy sauce, and ¼ tsp. Minced Ginger all placed atop a green salad; 1 can tuna, 1 Tbsp. mayonnaise (sugar free Canola Oil), chopped celery and green peppers; Chefs salad, blue cheese dressing; 5 oz. grilled swordfish, Pesto sauce (olive oil, pine nuts, basil, garlic and parmesian cheese), leafy green salad with flax oil dressing; 1 broiled lamb chop, asparagus, 1 Tbsp. grated mozarella; 1 lemon chicken breast, 1 oz. melted cheese, sauteed leeks in olive oil; 4 slices lean london broil, creamed spinach, 1 pat butter; 6 oz. turkey white meat, 2 Tbsp. Dijon mustard, 1 cup spinach salad, 2 tsp. olive and flax oil; ½ lb lean hamburger meat, 2 slice cheese, tomato and onion, 3 slices avocado; 6 oz. Can tuna, 2 Tbsp. Mayonnaise, Caesar salad, 3 slices avocado, 2 Tbsp. Caesar dressing; 6 oz breast grilled chicken with 1 Tbsp. pesto sauce, steamed spinach with rosemary, green salad with 2 Tbsp. flax oil dressing; 5 oz-wt broiled lamb chops, 1 cup cooked asparagus, spinach salad, 2 Tbsp. blue cheese dressing; 8 oz. swordfish or salmon, 1 cup broccoli, 2 butter pats, ½ cup grilled onions with 2 tsp. olive oil; 2 cups of salad with 2 oz. turkey, 2 oz. ham, 2 oz. of cheese, and lhardboiled egg, 3 Tbsp. caesar dressing, 2 Wasa crackers; o 5 oz. flank steak, ½ cup steamed cabbage, 2 pats of butter, mixed greens, 3 tsp. olive oil dressing.
Snack Choices: 4 macadamia nuts, 6 almonds, 1 Tbsp. ground flaxseeds on Wasa Crackers, hazelnuts, walnuts, brazil nuts, pumpkin seeds, or cashews, beef or turkey jerky (nitrate free), cheese cubes, cottage cheese, 2 celery sticks with 1 Tbsp. nut butter, protein shakes with freshly ground flaxseeds added, sugar-free yogurt sweetened with Xylitol, lean hormone free meat with mustard or horseradish, hard boiled egg or deviled egg, 1 Wasa sesame cracker with 1 oz of any cheese, roasted garlic or almond butter on celery, Smoked fish (salmon/lox).
Quick-n-easy meals: Ham and cheese salad; Broiled fish and vegetables; Cheeseburger (no bun); Poached egg; Tuna fish (no bread)
Beverages: (1): Green drinks: Greens First, Green Magma, Kyogreen, or Green Kamut: (1 tsp. 1-3× day in water); (2) Herbal Teas: Chamomile, Green Tea with Cinnamon Stick, Licorice, Ginger.
Avoid: Smoking, caffeine, sugar, hydrogenated oils, safflower, sunflower, corn oils, soft drinks, processed meats, refined or processed foods, hunger and craving producing carbohydrates—corn, beets, peas, carrots, potatoes, rice, grains, pasta & fruit
Suggestions And Goals: A healthful diet and lifestyle combined with key supplements is the best way to control hunger and cravings. Exercise regularly, reduce stress, and eat fresh whole foods consuming 25 grams of protein and 25 grams of carbohydrates with Breakfast, Lunch, and Dinner.

In some examples, bars and shakes such as the following might be recommended as part of the program:

Protein Shake: Calories 120, Calories from Fat, 20, Total Fat, 2 g, 3%*, Cholesterol, 20 mg, 7%, Sodium, 70 mg, 3%, Total Carbohydrate, 10 g, 3%*, Dietary Fiber, 3 g, 12%*, Sugars, 1 g, †, Protein (from Proserum® Whey), 17 g, 34%*, Vitamin C (as Sodium Ascorbate), 100 mg, 167%, Vitamin E (as d-alpha tocopherol), 15 IU, 50%, Thiamin (Vitamin B-1) (as Thiamin Mononitrate), 10 mg, 667%, Riboflavin (Vitamin B-2), 10 mg588%, Niacin (Vitamin B-3)(as Niacinamide), 10 mg, 50%, Vitamin B-6 (as Pyridoxine HCL), 10 mg, 500%, Folate (NatureFolate™ blend), 100 mcg, 25%, Vitamin B-12 (as Methylcobalamin), 50 mcg, 833%, Biotin (as d-Biotin), 100 mcg, 33%, Pantothenic Acid (as d-Calcium Pantothenate), 100 mg, 1000%, Calcium (from Proserum® Whey), 85 mg, 9%, Phosphorous (from Proserum® Whey), 60 mg, 6%, Magnesium (from Creatine MagnaPower®), 100 mg, 25%, Zinc (TRAACS® Zinc Glycinate Chelate), 5 mg, 33%, Chromium (TRAACS® Chromium Nicotinate Glycinate Chelate), 50 mcg, 42%, Potassium (from Proserum® Whey and natural vanilla flavor), 110 mg, 1%, Total Glutamine Value [Bound Glutamic Acid (Proserum® Whey) 3 g, L-Glutamine Value 900 mg], 3.9 g, †, Immunoglobulins (from Proserum® Whey) 2.1 g, †, Phosphatidylcholine (from soy lecithin), 840 mg, †, Creatine (from Creatine MagnaPower®), 550 mg, †, Conjugated Linoleic Acid (CLA), 120 mg, †, High Gamma Mixed Tocopherols (as d-gamma, d-delta, d-alpha, d-beta), 100 mg, †, Taurine, 100 mg, †, n-Zymes® (Amylase 2200 DU; Protease 4.5 5000 HUT; Protease 3.0 10.7 SAPU, Protease 6.0 [Concentrate] 2000 HUT; Lactase 400 ALU), 50 mg, †, Inositol, 50 mg.
Protein Bar: Coating (maltitol, cocoa butter, milk fat, sodium aseinate [milk] lethicin [soy], natural flavors, tocopherols), protein blend (whey protein isolate, whey protein concentrate, rice protein concentrate, whey crisps [whey protein concentrate, rice flour]), almond butter, glycerine, maltitol syrup, isomalto-oligosaccharides (prebiotic fiber), digestion-resistant fiber (Fibersol-2), coconut, MEG-3 Omega-3 powder (refined fish oil [anchovy, sardine], fish gelatin [tilapia]), soy lethicin, natural flavor, sea salt. Calories per bar: 180, Fat cal., 70, Total fat: 8 g, 12%, Sat. Fat: 4 g, 20%, Trans Fat: 0 g, Cholesterol: 10 mg, 3%, Sodium: 80 mg, 3%, Total carbs: 23 g, 8%, Fiber: 6 g, Sugars 1 g, Sugar alcohol 11 g, Protein 9 g.
Supplements such as the following for healthy leptin and ghrelin function to support healthy hunger and cravings might be recommended:

| Carnitine-Start Slow ¼ tsp. | 1,000-9,000 mg (½ to 1 hour | |
|---|---|---|
| | before breakfast and lunch) | |
| | (one teaspoon powder = 3 grams) | |
| Multi/Antioxidant | 2 AM/1 PM | |
| Vitamin A | 4,240 | IU |
| Vitamin C | 500 | mg |
| Vitamin D | 400 | IU |
| Vitamin E | 400 | IU |
| Vitamin K | 150 | mcg |
| Thiamin (B1) | 50 | mg |
| Riboflavin (B2) | 30 | mg |
| Niacin (B3) | 30 | mg |
| Vitamin B6 | 50 | mg |
| Folic Acid | 800 | mcg |
| Vitamin B12 | 100 | mcg |
| Biotin | 300 | mcg |
| Pantothenic Acid (B5) | 50 | mg |
| Calcium | 5 | mg |
| Iodine | 75 | mcg |
| Zinc | 15 | mg |
| Selenium | 200 | mcg |
| Copper | 2 | mg |
| Manganese | 3 | mg |
| Chromium | 400 | mcg |
| Molybdenum | 100 | mcg |
| Green Tea | 100 | mg |
| Citrus Bioflavonoid Complex | 100 | mg |
| Inositol | 75 | mg |
| Choline | 50 | mg |
| N-Acetyl Cysteine | 50 | mg |
| PABA | 50 | mg |
| Alpha Lipoic Acid | 50 | mg |
| Grape Seed Extract | 40 | mg |
| Silicon | 10 | mg |
| DMAE | 75 | mg |
| CoQ10 | 15 | mg |
| Gamma Oryzanol | 5 | mg |
| Boron | 3 | mg |
| Lutein | 3 | mg |
| Carotenoids | 3 | mg |
| Lycopene | 2 | mg |
| Vanadyl Sulfate | 100 | mcg |
| Calcium with Magnesium | 1 AM/1 PM | |
| Calcium Hydroxyapatite | 434 | mg |
| Calcium Carbonate | 257 | mg |
| Magnesium Citrate | 588 | mg |
| Magnesium Malate | 329 | mg |
| Magnesium Oxide | 88 | mg |
| Phosphorus | 56 | mg |
| Ethyl EPA | 2 capsules daily | |
| EPA | 300 | mg |
| DHA | 200 | mg |
| Vitamin E | 5 | IU |
| 5 HTP before meals | 50 mg 30 minutes | |
| Taurine | 1,000-2,000 | mg |
| African Mango | 150 | mg |
| (Irvingia gabonensis) | | |
| proprietary extract (seed) | | |
| Polysaccharides from acacia gum | 1400 | mg |
| and esterified fatty acids | | |

| | |
|---|---|
| Fiber | 1-3 Tbsp. of |
| | fiber(Ground Flaxmeal best) |

An exercise programs such as the following might be recommended to support healthy leptin and ghrelin function. There are many different types of “working out” in the exercise marketplace today, each designed to give you a different benefit or result. The goals of various types of exercise may be to increase muscle mass, increase strength, build cardiovascular conditioning, and cause weight loss. The recommended type of workout provides similar health benefits to that of cardio workouts, but offers some unique benefits not provided by any other form of exercise available. It is to boost your natural hormonal balance levels to give you the leaning, muscle building and metabolism boosting benefits of growth hormone, as well as balancing leptin and ghrelin to create a powerful anti-aging hormone balance that keeps one young, lean and healthy. When done correctly, this program will provide you with maximum health and esthetic benefits in the shortest amount of time spent exercising. Instead of hours upon hours of exercise, this exercise plan can be completed in 20 minutes and it falls under the category of high intensity training or high intensity interval training.

This exercise program is different from Cardiovascular (cardio for short) exercise—the popular form of exercise designed to improve endurance and stimulate fat loss while exercising. Cardio exercises include anything from long distance running, skipping, biking or even power walking. All of these forms of exercise fall under the category of moderate intensity training because they typically last anywhere from a half hour to more than an hour going at a constant pace (after a warm-up and followed by a cool down). While cardio exercise may burn calories while exercising and create some aerobic conditioning, the proposed exercise regimen is much more powerful at creating long term health benefits because it—
1. Burns calories for the 24 hours following its completion. Not just while exercising.
2. Builds lean body mass unlike aerobic exercise,
3. Provides both aerobic and anaerobic conditioning.
4. Increases metabolic rate to create a better metabolism,
5. Provides anti-aging benefits because of its ability to support healthy growth hormone, leptin and ghrelin levels.

### Clinical Studies Supporting Disclosed Exercise Program:

The benefits of high intensity interval training were discovered when a study compared this form of exercise to moderate intensity training (aerobic exercise). Tests were conducted on 2 groups of athletes; one of the groups used the moderate intensity interval training and the other using high intensity interval training. The athletes training with moderate intensity workouts (70% intensity) were performing five workouts a week for a total of six weeks with each training session lasting an hour. The high intensity group worked out for 4 days a week for a total of 6 weeks with each session lasting 4 minutes, at 20 seconds of intense training (170% intensity) and 10 seconds of rest.

### Results:

Group 1 had a significant increase in the aerobic system (cardiovascular system). However, the anaerobic system (muscles) gained little or no results at all. Calories were burned during the exercise session only. Group 2 showed much improvement in all their athletes. Their aerobic systems increased much more than group one, and their anaerobic systems increased by 28%. They gained muscle, increased their metabolic rate and burned calories for 24 hours following the exercise session resulting in greater leaning and fat loss.

### Conclusion:

Not only did high intensity interval training have more of an impact on the aerobic systems; it had an impact on the anaerobic systems, metabolic, and fat burning systems as well. The aerobic system uses oxygen to burn fuel, and the anaerobic system doesn't. What occurs with exercise is the human body starts out by burning fuel with your aerobic energy system, and once past the point where there is enough oxygen ones system to provide aerobic energy to the muscles, the anaerobic system kicks in. The goal of the disclosed exercise regimen is to create an oxygen debt. One will know when an oxygen debt is present by the subject panting heavily during exercise. This signals the body has burned off all of the blood sugar (glycogen) and is creating an energy deficit. This deficit is filled by burning fat. Many people erroneously believe that they want to try and burn fat/calories while exercising. This is incorrect. One wants to burn off carbohydrates as fuel when exercising so to cause the body to replace this fuel with fat/calorie burning after the exercise session. To make this happen, one needs to get their heart rate up past what is typically referred to as the ‘Target Heart Rate Zone’. One should use a Heart Rate Monitor to measure the heart rate while doing the exercise program. It is contemplated that one will start the program slowly and build it up over time. The long term benefit can include lower rates of cardiovascular disease, Type 2 diabetes, and cancer in addition to getting lean, muscular and increasing your metabolism and balancing your hunger, craving and satiety hormones. When done correctly, the benefits of this form of exercise far outweigh the calorie burning or cardiovascular benefits of other forms of exercise—such as aerobic exercise. The disclosed exercise program thus is to help boost hormonal balance through anaerobic exercise, which includes small bursts of intense exercise interspersed with periods of rest. The exercise program is to be performed for 20 minutes, 3 times a week. Each session which will include 8, 30-second sprints with 90 seconds of recovery in between. It is suggested to have the subject ease into this activity and not do all 8 sprints the first week.

The exercise regimen as follows:

1. Get a heart rate monitor. Maximum heart rate is 220−(age)=______
(if 30 years old, Max. HR would be 220−30=190 BPM—Beats Per Minute).
2. Take 2 grams of glutamine prior to this regimen.
3. Warm up for 5 minutes to get body temperature up (walk on the treadmill, elliptical, bike etc.).
4. To start the 20 minute session jog, bike, run or elliptical at a comfortable pace for 2 minutes, then start ones first sprint. All sprints are 30 seconds.
1st sprint—your goal is to achieve 50% max heart rate,
2^{nd} sprint 60% max heart rate,
3^{rd} sprint 70% max heart rate
4^{th} sprint 80% max heart rate
5^{th} sprint 90% max heart rate
6^{th}-8^{th} sprint 95% max heart rate
   Achieve this through biking, running, jump roping, elliptical, whatever exercise selected.
   5. After each sprint recover by exercise of choice at a slower rate for 90 seconds. In one example . . .
Warm up for 5 minutes
jog (or bike or elliptical or exercise of choice) for 2 minutes,
sprint for 30 seconds
walk or jog or slowly elliptical for 90 seconds,
sprint for 30 seconds,
walk or jog or slowly elliptical for 90 seconds
   8 sprints are to be done (when maximally fit) with 90 seconds of slower/rest exercise in between. If exercising right, one's heart rate go up a little after each sprint. This is because of the oxygen debt created, and it signals the body is trying to get more oxygen to your energy system. One will notice yourself panting—this is body's signal is trying to get more oxygen to your lungs to fuel your energy system. The first week do only 4 sprints. One will quickly develop exercise tolerance and have to work harder and harder to reach your max heart rate (this will happen faster than you think). When one notices at the end of their workout that their Recovery Heart Rate went down, add another sprint (bringing it to 5 per workout). The next time one notices a Recovery Heart Rate goes down after the workout from its previous number, increase the tension or intensity on the exercise equipment to make it more intense. Gradually, build up ones fitness level by first adding an interval, then increasing the tension. One will see your fitness level has improved from one workout to the next.

Once one becomes conditioned one will move closer and closer to doing ones first sprint at 70% and the majority of them at 95%. At 95% one should feel fatigued at 15 seconds into ones sprint and not be able to make it much beyond 30 seconds. Record maximum heart rate with each session. Cool down for 2 minutes and then completely rest (just sit or stand there) for 1 minute before resuming your post exercise daily activities. Record your heart rate again at this point. This is your Post Exercise Recovery heart rate. Drink water after each exercise session. Do not eat carbs for 2 hours after each exercise session because it will turn off the benefits of the session on hormone balance. One will know that they are successful at producing the desired effects if sweaty and warm afterward and muscles are slightly achy from lactic acid build-up. Do this workout regimen for 3 times per week to start. Allow at least one full day of recovery between workouts.

It is recommended that each subject check with their primary care physician before starting any kind of exercise routine to ensure that your physician does not find any contraindications to your ability to exercise safely without health risks.

### Example 2

This example provides a method for generating a personalized weight health profile and providing nutritional/diet/food products, supplements, food alternatives (shakes, bars, snacks), behavioral, lifestyle programs, a nutritional or dietary eating program or regimen recommendations and/or exercise regimens based upon the personalized weight health profile.

### Identification of SNPs

SNPs were identified through a search of SNPedia, the NCBI Variation Database (dsSNP), the HapMap Database as well as other scientific databases of SNP's and/or genetic markers to identify key factors/words and phrases that would reveal scientific information pertaining to biochemical factors that can affect or influence the ability to achieve an ideal weight. The resulting data and literature created from this search process was then reviewed to determine whether the biological significance of the identified polymorphisms or genetic markers was supported by scientific resources such as human studies and/or published peer-reviewed medical journals.

Criteria for the significance and scientific validity of SNPs included measuring their minor allele frequencies (MAFs, see below) which were verified in the dsSNP database and/or SNPedia, peer reviewed literature, or published human studies, and this information was used as criteria for inclusion or exclusion of the most relevant weight health related SNP's in the panel/profile. A PubMed search was carried out for each polymorphism and the frequency was determined. The resulting literature covered under each SNP was reviewed to determine the significance of the polymorphism supported by human studies, and/or it was verified in multiple human studies, and/or the results were published in peer-reviewed journals. Any SNP that was associated with at least one or more of the eight areas relevant to weight health was given consideration.

A process to cross-check the relevance, validity and significance of SNPs was used and included such things as their evaluation through the Genome Wide Association Studies (GWAS). After compiling a list of the most relevant weight health polymorphisms, the SNPs were grouped into one or more of the following categories:
1. Carbohydrate utilization or metabolism
2. Fat utilization or metabolism
3. Protein utilization or metabolism
4. Alcohol utilization or metabolism
5. Caffeine utilization or metabolism
6. Hunger and satiety hormones
7. Neurochemistry balance
8. Caloric utilization and exercise SNPs or any combination thereof

A process to prioritize SNPs in order of highest to lowest significance and relevance to weight health was done based on the quality and strength of the scientific data and relevance to their grouping. Abstracts, published peer-reviewed papers and other available literature were compiled as support material for the process and research carried out.

### Methodology for Maintaining Sample Integrity

To maintain sample integrity when analyzing the DNA, SNPs relevant to weight health for the disclosed system, a panel of one or more SNPs, possibly including a sex marker, were compiled that provide a DNA fingerprint for each individual. This process included methods of Cross et al (2009. Development of a fingerprinting panel using medically relevant polymorphisms. BMC Med Gen. 2:17, which is hereby incorporated by reference in its entirety). The criteria that were adapted from this method for the disclosed test included the following:
1. The polymorphisms may have a confirmed minor allele frequency (MAF) of 0.20. This methodology speaks to their significance and relevance in human populations.
2. The polymorphisms must be relevant in one or more areas of weight maintenance.
3. They must be easily detectable (Sequenom or other similar platform).

### Choosing a Lab for DNA Testing

After the SNP panel was finalized, a search was conducted to identify a lab to test the DNA samples. The lab obtained reagents such as primers from commercial resources such as Life Technologies that were capable of identifying the desired SNPs in human genomic DNA followed by confirmation that the SNPs in the panel were accurately verified by the testing method and reagents used.

### DNA Test Kit

The SNP panel was finalized, a lab was chosen to carry out the DNA analyses, and a test kit to obtain the DNA samples was designed that included:
1. One or more swabs for collecting the DNA sample from the inside of the cheek.
2. A questionnaire to be filled out by the individual.
3. An envelope into which the DNA swab is placed for mailing or delivering.
4. Instructions for collecting and submitting the sample.
5. A mailing envelope

### DNA Testing

The DNA testing process included the following:
1. A subject obtained the test by either purchasing online or a storefront (such as a healthcare provider or commercial entity that sells nutritional supplements).
2. The subject completed the questionnaire included in the DNA test kit to the best of their ability.
3. The subject removed the protected DNA test swabs from a sealed wrapper and rubbed the swabs one at a time across the inside of their cheek to obtain the DNA sample. They then, placed the swabs in the envelope provided, and filled out the questionnaire as per the instructions.
4. The individual provided the DNA sample (such as by mail or hand delivery) to Nutritional Medicine Associates.
5. Nutritional Medicine Associates forwarded the sample to the DNA testing lab.
6. The lab isolated the DNA and performed the testing.
7. A computer program was developed to weigh the statistical value of the DNA test results to create a report for the subject (as described in detail below).
8. The data was compiled and data/results were formatted into a DNA report.

### The DNA Report

The DNA report was statistically tabulated and electronically created through a computer system, and was comprised of the following:
1. A summary page that lists the overall risk or priority ratings of high, medium, or low under each of up to 8 or more categories of weight health.
2. A page describing the biological effects and the visible signs of each of the up to or more than 8 categories of weight health.
3. A page for each of the up to or more than 8 categories describing the category in greater detail and listing the marker-specific results for the individual is created. A rating system included the following:
   i. A value of 0.1 was assigned to a result of homozygous for the non-ideal allele at the at risk SNP location (non-ideal function), which indicated high risk and was represented by a deficient rating;
   ii. A value of 0.50 was assigned to a result of heterozygous for the non-ideal allele at the risk SNP location, which indicated medium risk and was represented by a sub-normal rating; and
   iii. A value of 1.00 was assigned to a result of homozygous for the ideal allele at the risk SNP location, which indicated low risk and was represented by a normal rating.
4. A computer program/algorithm calculated a numerical value for each category of weight health that was based on the average of the numbers provided for each SNP contained in a weight health category and generated an overall numerical rating.
5. High risk was considered an average numerical value of 0.1-0.5. Medium risk was considered to be a numerical average of 0.5-0.75 and low risk was considered to be a numerical value of 0.75-1.0.
6. Marker specific results were listed by SNP and included:
   The affected gene or marker
   The chromosomal location
   The ideal genotype
   The actual genotype
   The rating
7. Specific nutritional/diet/food products, supplements, food alternatives (shakes, bars, snacks), behavioral or lifestyle programs, nutritional or dietary eating program or regimen recommendations and/or exercise regimens for each individual were created according to a program based on each individual's report. Foundational products and programs were recommended for everyone and these did not represent the unique nutritional/diet/food products, supplements, food alternatives (shakes, bars, snacks), behavioral, lifestyle programs, nutritional or dietary eating program or regimen recommendations and/or exercise regimen recommendations made for the individual based on their genetic results. These products and programs are relevant; however, in that they set the stage for optimal results for personalized nutritional/diet/food products, supplements, food alternatives (shakes, bars, snacks), behavioral or lifestyle programs, nutritional or dietary eating program or regimen recommendations and/or exercise regimens based on each individual's unique DNA report. These foundational products and programs may have included:
   a. A Multi Vitamin
   b. An Antioxidant Supplement
   c. An Essential Fatty Acid Supplement
      Following recommendations for non-customized products and programs, one or more custom-designed nutritional/diet/food products, supplements, food alternatives (shakes, bars, snacks), behavioral or lifestyle programs, nutritional or dietary eating program or regimen recommendations and/or exercise regimens were recommended based on the DNA report. These recommendations address the individual strengths and weaknesses of the individual's ability to reach their ideal weight as determined by their DNA report.

The determination as to which nutritional/diet/food products, supplements, food alternatives (shakes, bars, snacks), behavioral or lifestyle programs, nutritional or dietary eating program or regimen recommendations and/or exercise regimens would be custom recommended was based on an algorithmic protocol that matched the nutritional/diet/food products, supplements, food alternatives (shakes, bars, snacks), behavioral or lifestyle programs, nutritional or dietary eating program or regimen recommendations and/or exercise regimens to the weaknesses that the person's DNA analysis revealed as identified in their medium or high risk or priority categories. This was programmed based on an algorithm into the computer system that generates the DNA report. High risk or priority categories were the most important category recommendations to follow as they provided the individual the greatest opportunity to improve healthy weight followed by the medium risk or priority category recommendations. For instance, if a person was determined to be medium or high risk or priority for hunger and satiety or cravings, nutritional/diet/food products, supplements, food alternatives (shakes, bars, snacks), behavioral and lifestyle programs, nutritional or dietary eating program or regimen recommendations and/or exercise regimens were recommended that supported healthy function and maintenance of hormones, neurochemicals, and other biochemical factors that regulated hunger, satiety, and cravings. The ingredients in the products that support healthy hunger, satiety, and craving control were selected and put into the healthy weight management products and nutritional supplements based on clinical research that supported the clinical effectiveness of the ingredients included in the product on the category to which it applies. Behavioral programs, nutritional or dietary eating program or regimen recommendations and/or exercise regimens were selected based on clinical research that supported the clinical effectiveness of the behavioral programs, nutritional or dietary eating program or regimen recommendations and/or exercise regimens in the category to which they apply. Nutritional/diet/food products, supplements, food alternatives (shakes, bars, snacks), behavioral programs, nutritional or dietary eating program or regimen recommendations and/or exercise regimen recommendations were organized into high and medium risk/priority categories on the report for ease of understanding and nutritional/diet/food products, supplements, food alternatives (shakes, bars, snacks), behavioral, lifestyle programs, nutritional or dietary eating program or regimen recommendations and/or exercise regimen selection by the individual. If a nutritional/diet/food product, supplement, food alternative (shakes, bars, snacks), behavioral lifestyle program, nutritional or dietary eating program or regimen recommendation and/or exercise regimen was already listed under a high-risk priority category, it was not relisted under a medium risk priority category as it was already selected by the algorithm and recommended.

Following the formulation process and algorithmic recommendations above, nutritional/diet/food products, supplements, food alternatives (shakes, bars, snacks), behavioral, lifestyle programs, nutritional or dietary eating program or regimen recommendations and/or exercise regimens were clinically used and evaluated in the inventor's clinical practice to ensure that the effectiveness, aesthetic appeal and client satisfaction are of the highest standards possible.

After reviewing the DNA report, the report was sent to the subject and an optional customer service meeting/call was set up for those who desired to go over the results and nutritional/diet/food products, supplements, food alternatives (shakes, bars, snacks), behavioral, lifestyle programs, nutritional or dietary eating program or regimen recommendations and/or exercise regimen recommendations, and/or had questions regarding their report with a Nutritional Medicine Associates representative.

Analysis of the data from this study confirmed the ability of the laboratory to accurately and reproducibly identify selected SNPs and verify their frequency in the population studied. Population frequencies were compared to the medical literature for accuracy. An algorithm was created to mathematically calculate the impact of each SNP on the applicable function. The example demonstrates that the disclosed methods provide clients/patients a highly personalized method by which they can objectively identify biological strengths and weaknesses in their genetic coding that affected their ability to reach their ideal or healthy weight. Further, the disclosed nutritional/diet/food products, supplements, food alternatives (shakes, bars, snacks), behavioral, lifestyle programs, nutritional or dietary eating program or regimen recommendations and/or exercise regimens and protocols demonstrate that the methods beneficially influence the targeted area of weight loss (or healthy weight).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

1. A method of characterizing a subject's weight, comprising generating a personalized weight profile by generating a personalized DNA report in which the personalized DNA report comprises an overall risk ratings for the likelihood of acquiring a deficiency in one or more areas of weight health, thereby revealing the subject's genetic strengths, weaknesses and/or risks related to the one or more areas of weight health thereby allowing a personalized weight, nutritional regimen and/or exercise regimen to be developed and implemented including nutritional/diet/food products, supplements, food alternatives (shakes, bars, snacks), behavioral or lifestyle programs, nutritional or dietary eating program or regimen recommendations and/or exercise regimens, wherein the rating system comprises assigning high risk to a result of homozygous for a non-ideal allele at an at risk single nucleotide polymorphism (SNP) location (non-ideal function); assigning medium risk to a result of heterozygous for the non-ideal allele at the risk SNP location; and assigning low risk to a result of homozygous for an ideal allele at the risk SNP location.

2. The method of claim 1, wherein generating a personalized DNA report comprises electronically creating the DNA report through use a computer system.

3. (canceled)

4. The method of claim 1, wherein the rating system comprises assigning a value of 0.1 to a result of homozygous for the non-ideal allele at an at risk single nucleotide polymorphism (SNP) location (non-ideal function); assigning a value of 0.50 to a result of heterozygous for the non-ideal allele at the risk SNP location; and assigning a value of 1.00 to a result of homozygous for the ideal allele at the risk SNP location; whereby high risk is considered an average numerical value of 0.1-0.5; medium risk is considered to be a numerical average of 0.5-0.75; and low risk is considered to be a numerical value of 0.75-1.0.

5. The method of claim 1, wherein the personalized DNA report further comprises a description of one or more biological effects and/or visible signs of the one or more areas of weight health.

6. The method of claim 1, wherein generating the personalized weight profile comprises determining a subject's genetic potential in at least one area of weight health by analyzing one or more weight health-associated single nucleotide polymorphisms (SNPs) or other genetic markers associated with the particular area of weight health being assessed in a biological sample obtained from the subject.

7. The method of claim 6, wherein the subject's genetic potential is determined by analyzing one or more weight health-associated SNPs or other genetic marker wherein the one or more weight health-associated SNPs or other genetic markers comprise at least one SNP or genetic marker associated with leptin, ghrelin and hunger, satiety or cravings, obesity or weight gain or loss related, carbohydrate, fat, protein and/or alcohol or caffeine utilization or metabolism, blood sugar regulation and stabilization, dopamine or neurochemistry, caloric utilization and exercise SNPs.

8. The method of claim 7, wherein the one or more weight health-associated molecular markers is a single nucleotide polymorphism (SNP) associated with leptin, ghrelin, hunger or satiety.

9. The method of claim 1, further comprising obtaining a biological sample from the subject prior to generating the personalized DNA report.

10. The method of claim 7, further comprising identifying one or more SNPs or other genetic markers associated with a particular area of weight health by searching the publically available SNP data bank and determining SNPs associated with particular weight conditions.

11. The method of claim 1, further comprising providing the generated weight profile to the subject and recommending and/or providing one or more weight treatments to the subject based upon the weight profile generated by the characterization analysis.

12. The method of claim 6, wherein the one or more weight health-associated SNPs comprise one or more of the SNPs listed in Table 1 and/or Table 2.

13. The method of claim 1, wherein generating the personalized weight profile comprises:
obtaining a genomic DNA sample from the subject;
isolating the genomic DNA from the sample;
determining the sequence of one or more weight health-associated SNPs in the genomic DNA;
inputting the sequence of one or more weight health-associated SNPs into a processing database;
performing group data analysis of the sequence of one or more weight health-associated SNPs to determine scores by weight health category; and
generating the personalized weight profile.
